(19) United States
(12) Patent Application Publication
(10) Pub. No.: US 2004/0170670 A1
(43) Pub. Date: Sep. 2, 2004

(54) Cosmetic sponges

(76) Inventors: James A. Smith, Chatham, MA (US); Wolfhard Scholz, Krefeld (DE); Heike Schelges, Krefeld (DE); Armin Wadle, Erkrath (DE); Bernhard Banowski, Dusseldorf (DE)

Correspondence Address: CONNOLLY BOVE LODGE & HUTZ, LLP, P O BOX 2207, WILMINGTON, DE, 19899, US

(21) Appl. No.: 10/469,759

(22) PCT Filed: Mar. 5, 2002
(86) PCT No.: PCT/EP02/02371

(30) Foreign Application Priority Data
Mar. 9, 2001 (DE) 101 11 689.6
Feb. 28, 2002 (DE) 102 08 678.8

(51) Int. Cl. A61K 9/70

(52) U.S. Cl. 424/443

(57) ABSTRACT

Disclosed herein are flexible, fine-pored sponges or foam pads impregnated with liquids. The foam pads are based on a polyurethane foam and may be used for the cosmetic and dermatological treatment of the skin, hair, mucosa and appendages of the skin.

### CROSS REFERENCE TO RELATED APPLICATIONS AND CLAIM FOR PRIORITY

The present application is a U.S. National Stage application filed under 35 U.S.C. § 371, claiming priority of International application No. PCT/EP02/02371, filed Mar. 5, 2002, and German Patent Application Nos. 101 11 689.6 and 102 08 678.8, filed Mar. 9, 2001 and Feb. 28, 2002, respectively, under 35 U.S.C. §§ 119 and 365.

### BACKGROUND OF THE INVENTION

A. Field of the Invention

The present invention relates generally to flexible, fine-pored sponges or foam pads impregnated with liquids. More particularly, the foam pads are based on a polyurethane foam and are obtained as in situ reaction products of a urethane prepolymer with free isocyanate groups and an aqueous phase, which contains at least one surface-active substance and cosmetic or dermatological active ingredients or care and maintenance ingredients. The foam pads are used for the cosmetic or dermatological treatment of the skin, hair, mucosa, and appendages of the skin.

B. Description of the Related Art

Flexible support materials, e.g. cloths and foam pads, that are saturated or impregnated with liquid cosmetic compositions, are extremely popular among consumers. They are time-saving, clean, and hygienic to use. The support materials often consist of nonwoven fleece, cotton, or microfibers.

Cosmetic foam pads based on in situ reaction products of a urethane prepolymer with free isocyanate groups and an aqueous phase are known in the art. For example, U.S. Pat. No. 4,806,572 describes foam pads, which are formed in situ from a urethane prepolymer with free isocyanate groups and an oil-in-water emulsion and are used for makeup removal. The emulsion contains anionic and nonionic surfactants and liquid oil components and is free of natural or synthetic waxes. U.S. Pat. No. 4,548,954 describes an oil-absorbing floor cleaner, which is formed in situ from a urethane prepolymer with free isocyanate groups and an aqueous silicone oil emulsion that contains abrasive particles. U.S. Pat. No. 4,127,515 describes a product for waxing and polishing floors, which is formed in situ from a urethane prepolymer with free isocyanate groups and an aqueous silicone oil emulsion that contains wax and a polyacrylate.

The advantage of in-situ foaming of the urethane prepolymer with the cosmetic or dermatological composition compared to conventional methods of impregnation is that it is a one-step process, i.e. the support material is impregnated with the liquid during the production process itself and does not have to be sprayed or impregnated with it in a second process step.

The foaming process and the quality of the final product depend to a great extent on the composition of the liquid that is used.

### SUMMARY OF THE INVENTION

The present inventors have found, surprisingly, that flexible, fine-pored foam pads may be obtained from the in situ reaction product of a urethane prepolymer with free isocyanate groups and a liquid aqueous phase that contains emulsifiers and cosmetic or dermatological active ingredients and care and maintenance ingredients, and that these foam pads have a pleasant slip on the skin and hair surfaces to be treated and allow controlled, drip-free deliver), of the liquid bound in the pores. The foams in accordance with the invention can be cured, cut, or punched into any desired shapes, preferably shapes adapted to the intended type of use.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### DETAILED DESCRIPTION OF EMBODIMENTS OF THE INVENTION

The following detailed description of the invention does not limit the invention. Instead, the scope of the invention is defined by the appended claims and equivalents thereof.

The present invention is broadly drawn to a flexible support material impregnated with liquid, which is intended for the cosmetic or dermatological treatment of the skin, hair, mucosa, and appendages of the skin. The flexible support material is based on a polyurethane foam that contains the in situ reaction product of a urethane prepolymer with free isocyanate groups and a liquid aqueous phase that contains at least one surface-active substance and at least one dispersed fatty substance. The present invention is characterized by the fact that the liquid phase additionally contains at least one cosmetic or dermatological active ingredient or care and maintenance ingredient, which is selected from among natural and, if desired, chemically modified polymers, which in turn are selected from among cellulose ethers, quaternized cellulose derivatives, polyquaternium 24, guar gum, cationic guar derivatives, alginates, xanthan gum, gum arabic, karaya gum, carob bean flour, linseed gums, dextrans, shellac, amylose, amylopectin, dextrins, chemically and/or thermally modified starches, and chitosan and its derivatives, as well as synthetic polymers that do not act as superabsorbers but rather swell with water and are thereby converted to a gel-like true or colloidal solution, α-hydroxycarboxylic acids and their derivatives, vitamins, provitamins and vitamin precursors of the groups B. C, and B and their derivatives, plant extracts, selected from among the plant formative tissues that are capable of dividing (meristem), green tea (Camellia sinensis), hamamelis, chamomile, marigold, pansy, peony, horse chestnut, sage, willow bark, cinnamon tree, chrysanthemums, oak bark, nettle, hops, lappa, horsetail, hawthorn, linden flowers, almonds, spruce needles, sandalwood, juniper, coconut, kiwi, guava, lime, mango, apricot, wheat, melon, orange, grapefruit, avocado, rosemary, birch, beech sprouts, mallow, cuckoo flower, yarrow, wild thyme, thyme, balm mint, restharrow, marsh mallow (Althaea), common mallow (Malva sylvestris) violet, leaves of the black currant, coltsfoot, cinquefoil, ginseng, ginger root, sweet potato, olives (Olea europaea) and citrus fruit seeds, extracts of algae and microorganisms, active substances with antiperspirant activity, selected from among astringent water-soluble aluminum, zinc, and zirconium salts and mixtures thereof, active substances with deodorant activity, silicic acids, natural and synthetic silicates, aluminosilicates, kaolin, talc, and apatites, which may be modified with aqueous carboxylic acids with 2-3 C atoms, pigments, selected from among the oxides of titanium, iron, zinc, zirconium, cerium, magnesium, and bismuth, which, if desired, may be surface-modified, boron nitride particles, water-insoluble nacreous pigments and water-insoluble organic pigments, water-soluble and oil-soluble organic sunscreens, cosmetic abrasives, selected from among polymer particles and vegetable abrasives, which, if desired, may be covered with fatty substances, dyes and oxidation dye (intermediates) for dyeing keratinous fibers, oxidizing agents, reducing agents, and active substances with sebum-regulating, skin-soothing, anti-inflammatory, astringent, or perfusion-promoting activity.

Urethane prepolymer compositions suitable for producing the foam pads of the invention are described, for example, in U.S. Pat. Nos. 3,903,232 and 4,137,200. Corresponding commercial products came from the Hypol® product line of W. R. Grace & Co., Lexington. MA, e.g., Hypol® FHP 5000, Hypol® FHP 4000, Hypol® FHP 3000, Hypol®FHP 2000, Hypol® FHP 2000 HD, Hypol® FHP 2002. Hypol® 2000. Hypol® 2002. Hypol® 3000, Hypol® X6100 and Hypol® Hydrogel, now offered by Dow Chemical Company. The liquid resins are produced by reacting polyols of low molecular weight and 3-8 hydroxyl groups with aromatic or aliphatic diisocyanates. After the reaction, the resins have at least two free isocyanate groups per molecule of polylol used. Examples of suitable diisocyanates are toluene diisocyanate, methylene diphenyl isocyanate and isophorone diisocyanate. Other suitable commercial products come from the Aquapol® product line of Freeman Chemical Corporation and the Trepol® product line of Twin Rivers Engineering.

When in contact with excess water from the liquid aqueous phase of the invention, which contains at least one surface-active substance and cosmetic or dermatological active ingredients and care and maintenance ingredients, the free isocyanate groups of the urethane prepolymer hydrolyze with evolution of carbon dioxide. This results in the formation of fine-pored foam pads impregnated with the aqueous phase, which contains the active substances.

The foamed material can then be converted to various shapes adapted to the specific type of intended application using conventional methods.

The surface-active substances used in accordance with the invention, which are called surfactants or emulsifiers, depending on the area of application of the substance, are selected from among anionic, cationic, dipolar-ionic (or zwitterionic), ampholytic, and nonionic surfactants and emulsifiers.

All anionic surface-active substances suitable for use on the human body are suitable for use as anionic surfactants and emulsifiers in preparations of the invention. These are characterized by an anionic group that makes the substances water-soluble, such as a carboxylate, sulfate, sulfonate, or phosphate group, and a lipophilic alkyl group with about 8-30 C (carbon) atoms. In addition, the molecule may contain glycol or polyglycol ether groups, ester, ether, and amide groups, and hydroxyl groups. Examples of suitable anionic surfactants and emulsifiers, each in the form of the sodium, potassium, and ammonium salts and the mono-, di-, and trialkanolammonium salts with 2-4 C atoms in the alkanol group are:

linear and branched fatty acids with 8-30 C atoms (soaps):

ether carboxylic acids with the formula R—O—(CH₂—CH₂O)—CH₂—COOH, in which R is a linear alkyl group with 8-30 C atoms, and x=0 or 1-16;

acyl sarcosides with 8-24 C atoms in the acyl group;

acyl taurides with 8-24 C atoms in the acyl group:

acyl isethionates with 8-24 C atoms in the acyl group;

acyl glutamates of formula (I)

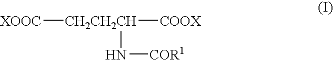

in which R¹CO is a linear or branched acyl group with 6-22 carbon atoms and 0, 1, 2, or 3 double bonds, and X is hydrogen, an alkali, and/or alkaline-earth metal, ammonium, alkylammonium, or glucammonium, for example, acyl glutamates derived from fatty acids with 6-22 and preferably 12-18 carbon atoms, such as C_{12/14} and C_{12/18} coco fatty acid, lauric acid, myristic acid, palmitic acid, and/or stearic acid, especially sodium N-cocoyl- and sodium N-stearoyl-L-glutamate;

esters of a hydroxy-substituted di- or tricarboxylic acid of general formula (II)

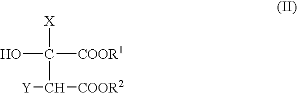

in which X is H or a CH₂COOR group: Y is H or —OH, under the condition that Y=H if X=CH₂COOR; and R, R¹, and R² are, independently of one another, a hydrogen atom, an alkali or alkaline-earth metal cation, an ammonium group, the cation of an ammonium organic base, or a group Z, which is derived from a polyhydroxylated organic compound selected from the group comprising etherified (C₆-C₁₈)alkyl polysaccharides with 1-6 monomeric saccharide units and/or etherified aliphatic (C₆-C₁₆)hydroxyalkyl polyols with 2-16 hydroxyl groups, subject to the condition that at least one of the groups R, R¹ or R², is a group Z;

esters of sulfosuccinic acid or sulfosuccinates of general formula (III)

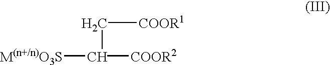

in which M^{(n+/n)} represents a hydrogen atom, an alkali metal cation, an ammonium group or the cation of an ammonium organic base for n=1 and an alkaline-earth metal cation for n=2 and R¹ and R² represent, independently of each other, a hydrogen atom, an alkali or alkaline-earth metal cation, an ammonium group, the cation of an ammonium organic base or a group Z, which is derived from a polyhydroxylated organic compound selected from the group comprising etherified (C₆-C₁₈) alkyl polysaccharides with 1-6 monomeric saccharide units and/or etherified aliphatic (C₆-C₁₆) hydroxyalkyl polyols with 2-16 hydroxyl groups, subject to the condition that at least one of the groups R¹ or R² is a group Z;

sulfosuccinic acid mono- and dialkyl esters with 8-24 C atoms in the alkyl group and sulfocuccinic acid monoalkyl polyoxyethyl esters with 8-24 C atoms in the alkyl group and 1-6 oxyethyl groups.

linear alkane sulfonates with 8-24 C atoms;

linear α-olefin sulfonates with 8-24 C atoms;

α-sulfo fatty acid methyl esters of fatty acids with 8-30 C atoms;

alkyl sulfates and alkyl polyglycol ether sulfates with the formula R—(O—CH₂—CH₂)ₓ—OSO₃H, in which R is preferably a linear alkyl group with 8-30 C atoms and x is 0 or 1-12;

mixed surface-active hydroxysulfonates in accordance with German Patent Application No. 37 25 030;

esters of tartaric acid and citric acid with alcohols, which represent addition products of about 2-15 molecules of ethylene oxide and/or propylene oxide to C₈₋₂₂ fatty alcohols;

alkyl and/or alkenyl ether phosphates;

sulfated fatty acid alkylene glycol esters; and

monoglyceride sulfates and monoglyceride ether sulfates.

Preferred anionic surfactants and emulsifiers are acyl glutamates, acyl isethionates, acyl sarcosinates, and acyl taurates, each with a linear or branched acyl group with 6-22 carbon atoms and 0, 1, 2, or 3 double bonds, which, in a preferred embodiment, is selected from among an octanoyl, decanoyl, lauroyl, myristoyl, palmitoyl, and stearoyl group, esters of tartaric acid, citric acid, or succinic acid and the salts of these acids with alkylated glucose, especially products with the INCI (International Nomenclature of Cosmetic Ingredients) names disodium coco glucoside citrates, sodium coco glucoside tartrates, and disodium coco glucoside sulfosuccinates, alkyl polyglycol ether sulfates, and ether carboxylic acids with 8-18 C atoms in the alkyl group and up to 12 ethoxy groups in the molecule, sulfosuccinic acid mono- and dialkyl esters with 8-18 C atoms in the alkyl group, and sulfosuccinic acid monalkyl polyoxyethyl esters with 8-18 C atoms and 1-6 ethoxy groups.

Surface-active compounds that contain at least one quaternary ammonium group and at least one —COO⁽⁻⁾ or —SO₃⁽⁻⁾ group in the molecule are refer-red to as dipolar-ionic surfactants and emulsifiers. Especially well-suited dipolar-ionic surfactants and emulsifiers are the so-called betaines, such as N-alkyl-N, N-dimethylammoniumn glycinates, for example, coco alkyl dimethylammonium glycinates, N-acylaminopropyl-N, N-dimethylammonium glycinates, for example, coco acyl aminopropyldimethylammonium glycinate, 2-alkyl-3-carboxymethyl-3-hydroxyethylimidazolines, in each case, with 8-18 C atoms in the alkyl or acyl group, and coco acylaminoethylhydryoxyethylcarboxyinethyl glycinate. A preferred dipolar-ionic surfactant is the fatty acid amide derivative with the INCI name cocamidopropyl betaine.

Ampholytic surfactants and emulsifiers are understood to mean those surface-active compounds that contain, in addition to a C₈-C₂₄ alkyl or acyl group, at least one free amino group and at least one —COOH or —SO₃H group and that are capable of forming inner salts. Examples of suitable ampholytic surfactants are N-alkyl glycines, N-alkylaminopropionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids, and alkylaminoacetic acids, each with about 8-24 C atoms in the alkyl group. Especially preferred ampholytic surfactants are N-coco alkylaminopropionate, coco acylaminoethylaminopropionate, and C₁₂-C₁₈acyl sarcosine.

Nonionic surfactants and emulsifiers contain, for example, a polyol group, a polyalkylene glycol ether group or a combination of a polyol and polyglycol ether group as the hydrophilic group. Examples of compounds of this type are:

addition products of 2-50 moles of ethylene oxide and/or 0-5 moles of propylene oxide to linear and branched fatty alcohols, with 8-30 C atoms, to fatty acids with 8-30 C atoms, and to alkyl phenols with 8-15 C atoms in the alkyl group;

C₁₂-C₃₀fatty acid mono- and diesters of addition products of 1-30 moles of ethylene oxide to polyols with 3-6 carbon atoms, especially to glycerol;

addition products of 5-60 moles of ethylene oxide to castor oil and hydrogenated castor oil;

polyol fatty acid (partial) esters, such as the Hydragen® HSP (Cognis) or Sovermol® types (Cognis), especially of saturated C₈-C₃₀ fatty acids;

alkoxylated fatty acid alkyl esters:

amine oxides,

fatty acid alkanolamides, fatty acid N-alkyl glucamides, and fatty amines and their ethylene oxide or polyglycerol addition products;

sorbitan fatty acid esters and addition products of ethylene oxide to sorbitan fatty acid esters, such as polysorbates;

sugar fatty acid esters and methyl glucoside fatty acid esters and their ethylene oxide or polyglycerol addition products, and;

alkyl polyglycosides of general formula RO-(Z)ₓ, in which R stands for an alkyl, Z stands for a sugar, and x stands for the number of sugar units. Especially preferred alkyl polyglycosides are those in which R consists essentially of:

C₈ and C₁₀ alkyl groups,

C₁₂ and C₁₄ alkyl groups,

C₈ to C₁₆ alkyl groups,

C₁₂ to C₁₆ alkyl groups,

C₁₆ to C₁₈ alkyl groups.

Any monosaccharides or oligosaccharides may be used as the sugar units. Sugars with 5 or 6 carbon atoms and the corresponding oligosaccharides are usually used. Examples of sugars of this type are glucose, fructose, galactose, arabinose, ribose, xylose, lyxose, allose, altrose, mannose, gulose, idose, talose, and sucrose. Preferred sugar units are glucose, fructose, galactose, arabinose, and sucrose, and glucose is especially preferred. The alkyl polyglycosides that can be used in accordance with the invention contain 1.1 to 5 sugar units on the average. Alkyl polyglycosides with values of x of 1.1 to 2.0 are preferred. Alkyl glycosides in which x is 1.1 to 1.8 are especially preferred.

Further examples of non-ionic surfactants and emulsifiers include:

mixtures of alkyl(oligo)glucosides and fatty alcohols, e.g. Montanov® 68;

sterols, e.g., ergosterol, stigmasterol, sitosterol and mycosterols;

phospholipids, e.g. lecithins and phosphatidal cholines;

polyglycerols and polyglycerol derivatives, such as polyglycerolpoly-12-hydroxystearate (Dehymuls® PGPH) or triglycerol diisostearate (Lameform® TGI); and

alkoxylated polydialkyl siloxanes (INCI name: dimethicone copolyol).

Nonionic surface-active substances that have been found to be preferable are alkyl polyglycosides, possibly mixed with fatty alcohols, alkoxylated polydialkyl siloxanes, alkylene oxide addition products with saturated linear fatty alcohols or fatty acids with, in each case, 2-30 moles of ethylene oxide per mole of fatty alcohol or fatty acid.

Furthermore, cationic surfactants of the following types may be used in accordance with the invention: quaternary ammonium compounds, esterquats, and amidoamines. Preferred quaternary ammonium compounds are ammonium halides, especially chlorides and bromides, such as alkyl trimethylammonium chlorides, dialkyl dimethyl ammonium chlorides, and trialkyl methylammonium chlorides. The lone alkyl chains of these surfactants preferably have 10-18 carbon atoms, such as cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Other preferred cationic surfactants are the imidazolium compounds known by the INCI names Quaternium 27 and Quaternium 83.

Esterquats are surface-active substances that contain both at least one ester functional group and at least one quaternary ammonium group as a structural element. Preferred esterquats are quaternized ester salts of fatty acids with triethanolamine, quaternized ester salts of fatty acids with diethanol alkylamifles, and quaternized ester salts of fatty acids with 1,2-dihydroxypropyl dialkylamines. The products Armocare® VGH 70, an N,N-bis(2-palmitoyloxyethyl)dimethylammonium chloride, and Dehyquart® F 75, Dehyquart® C 4046. Dehyquart® L 80, and Dehyquart® AU 35 are preferred examples of these types of esterquats.

The alkyl amidoamines are usually produced by amidation of natural or synthetic fatty acids and fatty acid cuts with dialkyl aminoamines. An especially well-suited compound from this class of substances is stearamidopropyl dimethylamine, e.g., the commercial product Tego Amide® S 18.

The surface-active substances are used in total amounts of 0.1-45 wt. %, preferably 0.1-30 wt. %, and most preferably 0.5-15 wt. %, based on the total weight of the composition used to form the foam pads of the invention.

The anionic surfactants and emulsifiers are preferably present in amounts of 0.1-30 wt. %, and especially in amounts of 1-15 wt. %, based on the total weight of the composition used to form the foam pads of the invention.

The dipolar-ionic (or zwitterionic) surfactants and emulsifiers are preferably present in amounts of 0.1-30 wt. %, and especially in amounts of 1-15 wt. %, based on the total weight of the composition used to form the foam pads of the invention.

The ampholylic surfactants and emulsifiers are preferably present in amounts of 0.1-15 wt. %, and especially in amounts of 1-10 wt. %, based on the total weight of the composition used to form the foam pads of the invention.

The nonionic surfactants and emulsifier-s are preferably present in amounts of 0.1-30 wt. %, and especially in amounts of 1-15 wt. %, based on the total weight of the composition used to form the foam pads of the invention.

The cationic surfactants are preferably present in amounts of 0.1-10 wt. %., and especially in amounts of 0.5-5 wt. %, based on the total weight of the composition used to form the foam pads of the invention.

The liquid, aqueous phases, with which the support materials of the invention are produced, preferably contain at least one nonionic emulsifier with an HLB value of 3-18 according to the definitions given in Römpp-Lexikon Chemie [Römpp's Chemical Dictionary] (Editors: J. Falbe, M. Regitz), 10th Edition, Georg Thieme Verlag, Stuttgart, New York (1997), p. 1,764. Nonionic o/w emulsifiers with an HLB value of 10-15 and nonionic w/o emulsifiers with an HLB value of 3-6 are especially preferred in accordance with the invention.

The fatty substances used in accordance with the invention are understood to be fatty acids, fatty alcohols, natural and synthetic waxes, which may be present in aqueous dispersion both in solid form and liquid form, and natural and synthetic cosmetic oil components. Perfume oils and essential oils are also regarded as fatty substances in accordance with the invention.

Linear and/or branched, saturated and/or unsaturated C₈₋₃₀ fatty acids may be used as fatty acids. C₁₀₋₂₂ fatty acids are preferred. Examples are isostearic acid and isopalmitic acid. Other suitable examples are caproic acid, caprylic acid, 2-ethyl hexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeosteric acid, arachidonic acid, gadoleic acid, behenic acid, and erucic acid, and their technical mixtures. The fatty acid cuts that can be obtained from coconut oil or palm oil are usually especially preferred, and the use of stearic acid is especially preferred.

The amount of fatty substance used is 0.1-15 wt. %, preferably 0.5-10 wt. %, and especially 1-5 wt. %, in each case, based on the total weight of the composition used to form the foam pads of the invention.

Fatty alcohols that may be used are saturated, mono- or polyunsaturated, branched or unbranched fatty alcohols with 6-30, preferably 10-22, and most preferably 12-22 carbon atoms. Examples of fatty alcohols that may be used in accordance with the invention are octenol, dodecenol, decenol, octadienol, dodecadienol, decadienol, oleyl alcohol, erucyl alcohol, ricinoleyl alcohol, stearyl alcohol, isostearyl alcohol, cetyl alcohol, lauryl alcohol, myristyl alcohol, arachic alcohol, caprylic alcohol, decyl alcohol, linoleyl alcohol, linolenyl alcohol, and behenyl alcohol, and their Guerbet alcohols.

Natural or synthetic waxes that may be used in accordance with the invention are solid paraffins or isoparaffins, vegetable waxes, such as candelilla wax, carnauba wax, esparto wax, fruit waxes and sunflower wax, beeswax and other insect waxes, ozocerites, ceresin, spermaceti, and microwaxes of polyethylene or polypropylene. Other suitable waxes are the triglycerides of saturated and possibly hydroxylated C₁₆₋₃₀ fatty acids, such as hydrogenated triglyceride fats (hydrogenated palm oil, hydrogenated coconut oil, hydrogenated castor oil), glyceryl tribehenate or glyceryl tri-12-hydroxystearate synthetic complete esters from fatty acids and glycols (e.g., Syncrowax®) or polyols with 2-6 C atoms, fatty acid monoalkanolamides with a C₁₂₋₂₂ acyl group and a C₂₋₄ alkanol group, synthetic fatty acid-fatty alcohol esters. e.g. stearyl stearate or cetyl palmitate, ester waxes from natural fatty acids and synthetic C₂₀₋₄₀fatty alcohols (INCI designation: C₂₀₋₄₀ alkyl stearates), and complete esters from fatty alcohols and di- and tricarboxylic acids, e.g., dicetyl succinate or dicetyl/stearyl adipate and mixtures of these substances.

The natural and synthetic cosmetic oil components that may be advantageously used in accordance with the invention include, for example:

vegetable oils. Examples of vegetable oils are sunflower oil, olive oil, soybean oil, rapeseed oil, almond oil, jojoba oil, orange oil, wheat germ oil, peach kernel oil, and the liquid components of coconut oil. However, other triglyceride oils are also suitable, such as the liquid components of beef tallow and synthetic triglyceride oils;

liquid paraffin oils, isoparaffin oils, and synthetic hydrocarbons, such as 1,3-di(2-ethylhexyl)cyclohexane (Cetiol® 5) and di-n-alkyl ethers with a total of 12-36, and especially 12-24 C atoms, such as di-n-octyl ether, di-in-decyl ether, di-n-nonyl ether, di-n-undecyl ether, n-hexyl-n-octyl ether, and n-octyl-in-decyl ether. 1,3-Di(2-ethylhexyl)cyclohexane (Cetiol® (S) and di-n-octyl ether (Cetiol® OE) may be preferred:

ester oils. Ester oils are understood to be the esters of C₆₋₃₀fatty acids with C₂₋₃₀fatty alcohols. The monoesters of the fatty acids with alcohols with 2-24 C atoms are preferred. The following are especially preferred in accordance with the invention: isopropyl myristate, isononanoic acid C₁₆₋₁₆ alkyl esters, 2-ethylhexyl palmitate, 2-ethylhexyl stearate, cetyl oleate, glycerol tricaprylate, coco fatty alcohol caprate/caprylate, n-butyl stearate, oleyl erucate, isopropyl palmitate, oleyl oleate, hexyl laurate, di-n-butyl adipate, myristyl myristate, cetearyl isononanoates, and decyl oleate;

dicarboxylic acid esters, such as di-n-butyl adipate, di(2-ethylhexyl) adipate, di(2-ethylhexyl) succinate, and diisotridecyl acelaate, as well as diol esters, such as ethylene glycol dioleate, ethylene glycol diisotridecanoate, propylene glycol di(2-ethylhexanoate), propylene glycol diisostearate, propylene glycol dipelargonate, butanediol diisostearate, and neopentyl glycol dicaprylate;

symmetrical, unsymmetrical, or cyclic esters of carbon dioxide with fatty alcohols, as described, for example, in German Patent No. 197 56 454, glycerol carbonate, or dicaprylyl carbonate (Cetiol® CC); and

mono-, di-, and tri-fatty acid esters of saturated and/or unsaturated linear and/or branched fatty acids with glycerol, e.g., Monomuls® 90018, Monomuls® 90L12, or Cutina® MD.

The amount of natural and synthetic cosmetic oil components used is 0.1-50 wt. %, preferably 0.1-20 wt. %, and especially 0.1-15 wt. %, based on the total weight of the cosmetic composition used to form the foam pads of the invention.

Other oils that can be advantageously used in accordance with the invention are hydroxycarboxylic acid esters. Hydroxycarboxylic acid esters are complete esters of glycolic acid, lactic acid, malic acid, tartaric acid, or citric acid. Other hydroxycarboxylic acid esters that are basically suitable are esters of β-hydroxypropionic acid, tartronic acid, D-gluconic acid, D-gluconic acid, mucic acid, or glucuronic acid. Primary linear or branched aliphatic alcohols with 8-22 C atoms are suitable as the alcohol component of these esters, and the esters of C₁₂₋₁₅ fatty alcohols are especially preferred. Esters of this type can be obtained, for example, under the trade name Cosmacol® (Eni Chem. Augusta Industriale). Other especially preferred oil components are the esters of C₁₂₋₁₃alkanols branched at the 2-position with 2-ethyl hexanoic acid. e.g. the commercial product Cosmacol® EOI. The amount of hydroxycarboxylic acid esters used is 0.1-15 wt. %, preferably 0.1-10 wt. %, and especially 0.1-5 wt. %, in each case, based on the total weight of the composition used to form the foam pads of the invention.

Other fatty substances that can be advantageously used in accordance with the invention are siloxanes. The siloxanes may be present as oils, resins, elastomers, or gums. Preferred siloxanes are polydialkyl siloxanes, e.g., polydimethylsiloxane, polyalkylanyl siloxanes, e.g. polyphenylmethylsiloxane, polydialkyl siloxanes that contain amino and/or hydroxyl groups, and cyclic silicones (INCI name: cyclomethicones), preferably decamethylcyclopentasiloxane and dodecamethylcyclohexasiloxane.

Suitable perfume oils are, for example, mixtures of natural and synthetic fragrances. Natural fragrances are extracts of flowers (lily, lavender, roses, jasmine, neroli, ylang-ylang), stems and leaves (geranium, patchouli, petitgrain), fruits (anise, coriander, caraway, juniper), fruit skins (bergamot, lemon, oranges), roots (mace, angelica, celery, cardamom, costus, iris, calamus), woods (pines, sandalwood, guaiac, cedar, rosewood), herbs and grasses (estragon, lemongrass, sage, thyme), needles and twigs (spruce, fir, pine, dwarf pine), resins and balsams (galbanum, elemi, benzoin, myrrh, olibanum, opoponax). Raw materials of animal origin may also be used, for example, civet and castoreum. Typical synthetic fragrant compounds are products of the following type: esters, ethers, aldehydes, ketones, alcohols, and hydrocarbons. Examples of fragrant compounds of the ester type are benzyl acetate, phenoxyethyl isobutyrale, p-tert-butylcyclohexyl acetate, linalyl acetate, dimethylbenzylcarbinyl acetate, phenylethyl acetate, linalyl benzoate, benzyl format, ethylmethylphenyl glycinate, allylcyclohexyl propionate, styralyl propionate, and benzyl salicylate. The ethers include, for example, benzyl ethyl ether. The aldehydes include, for example, linear alkanals with 8-18 C atoms, citral, citronellyl, citronellyl oxyacetaldehyde, cyclamen aldehyde, hydroxycitronellal, lilial, and bourgeonal. The ketones include, for example, ionones, α-isomethylionone, and methyl cedryl ketone. The alcohols include, for example, anethol, citronellol, eugenol, isoeugenol, geraniol, linalool, phenylethyl alcohol, and terpineol. The hydrocarbons include mainly the tenpenes and balsams. Preferably, however, mixtures of different fragrances are used, which together produce a pleasing fragrance.

Essential oils of low volatility, which are usually used as flavoring components, are also suitable as perfume oils, e.g., sage oil, chamomile oil, oil of cloves, balm mint oil, spearmint oil, cinnamon leaf oil, linden flower oil, juniper berry oil, vetiver oil, olibanum oil, galbanum oil, labolanum oil, and lavender oil.

The use of the following, alone or in mixtures, is preferred: bergamot oil, dihydromyrcenol, lilial, lyral, citronellol, phenylethyl alcohol, α-hexylcinnamaldehyde, geraniol, benzyl acetone, cyclamen aldehyde, linalool, boisambrene forte, ambroxan, indole, hedione, sandelice, lemon oil, mandarin oil, orange oil, allylamyl glycolate, cyclovertal, lavender oil, muscatel sage oil, β-damascone, geranium oil bourbon, cyclolhexyl salicylate, vertofix coeur, Iso-E Super, Fixolide NP, evernyl, iraldein gamma, phenylacetic acid, geranyl acetate, benzyl acetate, rose oxide, romilllate, irotyl, and floramat.

In accordance with the invention, the perfume oil and/or essential oil is present in amounts of 0.01-2 wt. %, and preferably 0.1-1 wt. %, in each case, based on the total weight of the composition used to form the foam pads of the invention.

The total amount of oil components and fatty substances present is usually 0.01-60 wt. %, preferably 0.1-35 wt. %, and especially 1-20 wt. %, in each case, based on the total weight of the composition used to form the foam pads of the invention.

The preferred natural, if desired, chemically modified, polymers of the invention include cellulose ethers, e.g. hydroxypropyl cellulose, carboxymethyl cellulose, hydroxyethyl cellulose, and methylhydroxypropyl cellulose, quaternized cellulose derivatives, e.g. the commercial products Celquat® and Polymer JR®, and especially Celquat® H 100. Celquat® L 200, and Polymer JR® 400, polymers known under the name polyquaternium 24, guar gum, cationic guar derivatives especially the products Cosmedia® Guar and Jaguar® alginates, xanthan gum, gum arabic, karaya gum, carob bean flour, linseed gums, dextrans, shellac, starch fractions, such as amylose, amylopectin, and dextrins, chemically and/or thermally modified starches, e.g., aluminum starch octenyl succinate (Dry Flo® Plus) and hydroxypropyl starch phosphate, chitosan and its derivatives, e.g., the products Hydragen® CMF, Hydragen® HCMF. Kytamer® PC, and Chitolam® NB/101. Chitosans that are especially Well suited have a degree of deacelylation of at least 80% and a molecular weight of 5×10⁵ to 5×10⁶ g/mole. The chitosan must be converted to a salt to be used in accordance with the invention. Examples of suitable acids for this purpose are acetic acid, glycolic acid, tartaric acid, malic acid, citric acid, lactic acid, 2-pyrrolidone-5-carboxylic acid, benzoic acid, or salicylic acid.

Preferred synthetic polymers for use in accordance with the invention are synthetic polymers that do not act as super-absorbers but rather swell with water and are thereby converted to a gel-like true or colloidal solution and may be anionic, cationic, amphoteric, or nonionic.

Suitable anionic synthetic polymers contain carboxylate and/on sulfonate groups and, as monomers, e.g., acrylic acid, methacrylic acid, crotonic acid, maleic anhydride, and 2-acrylamido-2-methylpropanesulfonic acid. In this regard, the acid groups may be present wholly or partially as sodium, potassium, ammonium, or mono- or triethanolammonium salt. Preferred monomers are 2-acrylamido-2-methylpropanesulfonic acid and acrylic acid. Especially preferred anionic polymers contain 2-acrylamido-2-methylpropanesulfonic acid as the sole monomer or as a comonomer, and the sulfonic acid group may be present wholly or partially in the form of its salt. The homopolymer of 2-acrylamido-2-methylpropanesulfonic acid. e.g. the product Rheothik® 80-11 is especially preferred.

Within this embodiment, it may be preferable to use copolymers of at least one anionic monomer and at least one nonionic monomer. With respect to the anionic monomers, see the substances listed above. Preferred nonionic monomers are acrylamide, methacrylamide, acrylates, methacrylates, vinyl pyrrolidone, vinyl ethers, and vinyl esters. Preferred anionic copolymers are acrylic acid-acrylamide copolymers and especially polyacrylamide copolymers with monomers that contain sulfonic acid groups. An especially preferred anionic copolymer consists of 70-55 mole % acrylamide and 30-45 mole % 2-acrylamido-2-methylpropanesulfonic acid, in which the sulfonic acid groups are present, wholly or partially, in the form of the sodium, potassium, ammonium, or mono- or triethanolammonium salt. This copolymer may also be present in crosslinked form. Preferred crosslinking agents are polyolefinically unsaturated compounds, such as tetraallyl oxyethane, allyl sucrose, allyl pentaerythritol, and methylenebisacrylamide. A polymer of this type is contained in the commercial product Sepigel® 305 of the company SEPPIC. The use of this compound has proven especially effective in the context of the teaching of the invention. Sodium acryloyl dimethyltaurate copolymers sold under the trade name Simulgel® 600 as a compound with isohexadecane and polysorbate 80 have also proven especially effective in accordance with the invention.

Crosslinked and uncrosslinked polyacrylic acids are also preferred anionic homopolymers. Allyl ethers of pentraenithritol, of sucrose, and of propylene may be preferred crosslinking agents. Compounds of this type include, for example, the products sold under the name Carbopol®. Other especially preferred anionic copolymers are those that contain as monomers 80-98 wt. % of possibly substituted acrylic acid and 2-20 wt. % of C₁₂₋₃₀fatty alcohol methacrylic acid esters and may be crosslinked. The commercial products Permulen® are examples of compounds of this type.

Amphoteric polymers are understood to include both polymers that contain both free amino groups and free —COOH or SO₃H groups in the molecule and are capable of forming inner salts and dipolar-ionic polymers that contain quaternary ammonium groups and —COO⁻ or SO₃⁻ groups or —COOH or SO₃H groups. An example of an amphoteric polymer that can be used in accordance with the invention is the acrylic resin sold under the name Amphomer®, which is a copolymer of tert-butylaminoethyl methacrylate, N-(1,1,3,3-tetramethylbutyl)acrylamide and two or more monomers from the group comprising acrylic acid, methacrylic acid, and its esters.

The present invention may also contain nonionic polymers. Examples of suitable nonionic polymers are:

polyvinyl pyrrolidones and vinyl pyrrolidone/vinyl ester copolymers. e.g., the commercial products Luviskol® (BASF): and

polyvinyl alcohols, which may be partially saponified.

Cationic polymers that are suitable in accordance with the invention are, for example, polysiloxanes with quaternary groups, e.g., the commercial products Q2-7224 (Dow Corning). Dow Corning® 929 Emulsion, SM-2059 (General Electric). SLM-55067 (Wacker), and Abil®-Quat 3270 and 3272 (Th. Goldschmidt) as well as the polymers with quaternary nitrogen atoms in the main chain of the polymer that are known by the names polyquaternium 2, polyquaternium 17, polyquaternium 18, and polyquaternium 27.

A cationic polymer that is preferred in accordance with the invention is poly(methacryloyl oxyethyltrimethylammonium chloride) with the INCI name polyquaternium 37, which, if desired, may be crosslinked. The crosslinking can be carried out with polyolefinically unsaturated compounds, for example, divinylbenzene, tetraallyl oxyethane, methylenebisacrylamide, diallyl ether, polyallyl polyglyceryl ether, or allyl ethers of sugars or sugar derivatives, such as erithritol, pentaerythritol, arabitol, mannitol, sorbitol, sucrose, or glucose. Methylenebisacrylamide is a preferred crosslinking agent. Polyquaternium 37 is preferably used in the form of a nonaqueous polymer dispersion. Polymer dispersions of this type can be obtained under the names Salcare® SC 95 and Salcare® SC 96.

Copolymers of methacryloyl oxyethyltrimethylammonium chloride and nonionic monomers, preferably acrylamide methacrylamide: acrylic acid C₁₋₄-alkyl esters, and methacrylic acid C₁₋₄-alkyl esters, which may possibly be crosslinked, are commercially available under the name Salcare® SC 92.

In accordance with the invention, it is also possible that the cosmetic compositions used to form the foam pads of the invention contain several, especially two, different polymers of the same charge and/or one ionic and one amphoteric and/or nonionic polymer.

In another preferred embodiment, the compositions used to form the foam pads of the invention contain α-hydroxycarboxylic acids. In accordance with the invention, the α-hydroxycarboxylic acids are selected from among the following glycolic acid, lactic acid, methyl lactic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid (α-hydroxylauric acid), 2-hydroxytetradecanoic acid (α-hydroxymyristic acid), 2-hydroxyhexadecanoic acid (α-hydroxypalmitic acid), 2-hydroxyoctadecanoic acid (α-hydroxystearic acid) 2-hydroxyeicosanoic acid (α-hydroxyanachidonic acid), mandelic acid, phenyllactic acid, glyceric acid, 2,3,4-trihydroxybutanoic acid with the isomers erythonic acid and threonic acid, ribonic acid, arabinonic acid, xylonic acid, lyxonic acid, allonic acid, allonic acid, altronic acid, gluconic acid, mannonic acid, gulonic acid, idonic acid, galactonic acid, talonic acid, glucoheptonic acid, galactoheptonic acid, tartronic acid, malic acid, tartaric acid, mucic acid (galactaric acid), glucaric acid, and the physiologically tolerated salts of the above acids and their lactone forms, especially gluconolactone, galactolactone, glucuronolactone, galacturonolactone, gulonolactone, ribonolactone, glucoheptonolactone, mannonolactone, galactoheptonolactone and pantoyllactone.

Citric acid is not related to the alpha-hydroxycarbonic acids according to the invention.

The alpha-hydroxycarbonic acids are used in amount of 0.01-10 wt. %, preferably 0.1-5 wt. %, most preferably 1-3 wt. % based on the total weight of the composition used to form the foam pads of the invention.

According to the invention, vitamins provitamins and pre-vitamins of the groups B, C and H can be used as the appropriate active agents.

The vitamins of B-group or vitamins B-complex are:

vitamin B1 (thiamin);

vitamin B2 (riboflavin);

vitamin B3. This name is used mainly for the compounds of nicotinic acid and nicotinic acid amide (Niacinamid). According to the invention, the nicotinic acid amide is preferable, and content thereof is of 0.05 to 1 wt. % based on the total weight of the composition used to form the foam pads of the invention; and

vitamin B5 (pantothenic acid and panthenol). Preferably, panthenol is used.

According to the invention, the most preferable derivatives of panthenol are the esters and ethers thereof and also the cationic derivatives thereof. According further preferable embodiment of the invention, a derivate of 2-furanone having a general structural formula (1):

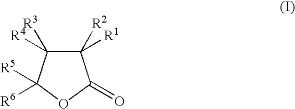

can be used instead or additionally to pantothenic acid or panthenol. The preferable furanone derivates are those ones where substituents R¹ to R⁶, independently of each other, can be hydrogen, hydroxy-, methyl-, methoxy-, aminomethoxy- or hydroxymethyl-rest, a saturated or one- or two-unsaturated linear or branched C₂-C₄ hydrocarbon group, a saturated or one- or two-unsaturated linear or branched mono- , di- or trihydroxy-C₂-C₄ hydrocarbon group, or a saturated or one- or two-unsaturated linear or blanched mono-, di- or triamino-C₂-C₄ hydrocarbon group. The most preferable derivates are those available on the market as dihydro-3-hydroxy-4,4-dimethyl-2(3H)-furanon having the generic name Pantolacton (Merck), 4-hydroxymethyl-gamma-butyrolacton (Merck), 3,3-dimethyl-2-hydroxy-gamma-butyrolacton (Aldrich) and 2,5-dihydro-5-methoxy-2-furanon (Merck), wherein expressly all the stereoisomers are included. According to the invention, the most preferable derivate is Pantolacton (dihydro-3-hydroxy-4,4-dimethyl-2(3H)-furanon), and, regarding formula (I), R¹ is the hydroxy-group, R² is the hydrogen atom, R³ and R⁴ each is the methyl group, and R⁵ and R⁶ each is the hydrogen atom. The stereoisomer (R)-Pantolacton is formed by degradation of pantothenic acid.

The mentioned compounds of the vitamin B6 type and also 2-furanon derivate are preferably provided in a total amount of 0.05 to 10 wt. %, and more preferably 0.1 to 5 wt. %, based on the total weight of the composition used to form the foam pads of the invention.

The vitamins of B-group or vitamins B-complex group further may include:

vitamin B6 (pyridoxin and also pyridoxamin and pyridoxal); and

vitamin B7 (Biotin), also known as Vitamin H or “Skin vitamin”.

Preferably, Biotin is provided in an amount of 0.0001 to 1.0 wt. %, especially 0.001 to 0.01 wt. %, based on the total weight of the composition used to form the foam pads of the invention.

Vitamin C (ascorbic acid) or derivates thereof is used preferably in amount of 0.1 to 3 wt. %, based on the total weight of the composition used to form the foam pads of the invention. The glucoside or phosphate may be preferable when used in a form of palmitinic acid ester, glucoside or phosphate. A combination with tocopherols may also be preferable to form the foam pads of the invention. Use in the form of the palmitic acid ester, glucosides, or phosphates may be preferred. Use in combination with tocopherols may also be preferred.

Preferred vitamins, provitamins, and vitamin intermediates in the groups B, C, and H are panthenol and its derivatives, pantolactone, nicotinic acid amide, ascorbyl palmitate, sodium ascorbyl phosphate, magnesium ascorbyl phosphate, and biotin.

Plant extracts that are suitable in accordance with the invention are selected from among plant formative tissues that are capable of dividing (menistem), green tea (Camellia sinensis), hamamelis, chamomile, marigold, pansy, peony, horse chestnut, sage, willow bark, cinnamon tree, chrysanthemums, oak bark, nettle, hops, lappa, horsetail, hawthorn, linden flowers, almonds, spruce needles, sandalwood, juniper, coconut, kiwi, guava, lime, mango, apricot, wheat, melon, orange, grapefruit, avocado, rosemary, birch, beech sprouts, mallow, cuckoo flower, yarrow, wild thyme, thyme, balm mint, restharrow, marsh mallow (Althaea), common mallow (Malva sylvestris), violet, leaves of the black currant, coltsfoot, cinquefoil, ginseng, ginger root, sweet potato, olives (Olea europaea), especially olive tree leaves and citrus fruit seeds, especially from the seeds of Citrus sinensis, C. paradisi, C. aurantium, C. aurantifolia. C. reticulata, C. grandis, C. limonia, and C. medica. They are usually produced by extraction of the whole plant, but in individual cases, they are also produced exclusively from flowers and/or leaves and/or seeds and/or other plant parts.

It may also be advantageous to use algae extracts. The algae extracts used in accordance with the invention are derived from green algae, brown algae, red algae, or blue-green algae (cyanobacteria). The algae used for the extraction may be obtained both from natural sources and by bioengineering processes and, if desired, may be modified from the natural form. The organisms may be modified by genetic engineering, by culture or by cultivation in media enriched with selected nutrients. Preferred algae extracts are derived from seaweed, blue-green algae, from the green alga Codium tomentosum, and from the brown alga Fucus vesiculosus. An especially preferred algae extract is derived from blue-green algae of the species Spirulina cultivated in a magnesium-enriched culture medium.

Extracts of microorganisms may also be advantageously used, e.g., from yeasts, preferably baker's yeast.

Especially preferred are extracts of Spirulina, baker's yeast, green lea (Camellia sinensis), meristem, hamamaelis, apricot, marigold, guava, sweet potato, lime, mango, kiwi, cucumber, mallow, marsh mallow, violet, olive tree leaves, and Citrus sinensis. The present invention may also contain mixtures of several, especially two, different plant extracts.

Suitable antiperspirant substances in accordance with the invention are water-soluble, astringent metallic salts, especially inorganic and organic salts of aluminum, zirconium, and zinc or any desired mixtures of these salts. Examples of substances that may be used in accordance with the invention are alum (KAl(SO₄)₂.12H₂O), aluminum sulfate, aluminum lactate, sodium aluminum chlorohydroxylactate, aluminum chlorohydroxy allantoinate, aluminum chlorohydrate, aluminum sulfocarbolate, aluminum zirconium chlorohydrate, zinc chloride, zinc sulfocarbolate, zinc sulfate, zirconium chlorohydrate, and aluminum zirconium chlorohydrate-glycine complexes. In accordance with the invention, solubility in water is defined as a solubility of at least 5 g of active substance per 100 g of solution at 20° C. The antiperspirant active substances are used as aqueous solutions. In accordance with the invention, they are contained in an amount of active substance of 1-40 wt. %, preferably 5-30 wt. %, and especially 10-25 wt. %, based on the total weight of the composition used to form the foam pads of the invention. In a preferred embodiment, the composition contains an astringent aluminum salt, especially aluminum chlorohydrate, and/or an aluminum-zirconium compound. Aluminum chlorohydrates are sold, for example, in powdered form as Micro Dry® Ultrafine by Reheis, in the form of an aqueous solution as Locron® L by Clariant, as Chlorhydrol®, and in activated form as Reach® 501 by Reheis. Reheis offers an aluminum sesquichlorohydrate under the name Reach® 301. The use of aluminum zirconium tetrachlorohydrex-glycine complexes, which are commercially available under the name Rezal® 36G from Reheis, is also especially advantageous in accordance with the invention.

Suitable deodorants in accordance with the invention are fragrances, antimicrobial, antibacterial, or bacteriostatic substances, enzyme-inhibiting substances, antioxidants, and odor-adsorbents.

Suitable antimicrobial, antibacterial, or bacteriostatic substances include especially organohalogen compounds and organiohalides, quaternary ammonium compounds, a series of plant extracts, and zinc compounds. Preferred compounds are halogenated phenol derivatives, e.g. hexachlorophene or Irgasan DP 300 (triclosan, 2,4,4′-trichloro-2′-hydroxydiphenyl ether), 3,4,4′-trichlorocarbonilide, chlorhexidine (1,1′-hexamethylenebis [5-(4-chlorophenyl) biguanide), chlonhexidine gluconate benzalkonium halides, bromochlorophene, dichlorophene, chlorothymol, chloroxylenol, hexachlorophene, cloflucarban, dichloro-m-xylenol, dequalinium chloride, domiphen bromide, ammonoium phenolsulfonate, benzalkonium halides, benzalkonium cetyl phosphate, benzalkonium saccharinates, benzethoniumn chloride, lauryl pyridinium chloride, lauryl isoquinolinium bromide, cetyl pyridinium chloride, and methyl benzedonium chloride. Plant extracts with antimicrobial activity that are especially preferred in accordance with the invention are selected from among water-soluble and oil-soluble extracts of the leaves of the black currant, chamomile flowers, cloves, lappa, pansy, ribgrass, Citrus sinensis, and green tea (Camellia sinensis), and from among the terpene alcohols, e.g. farnesol, and components of linden flower oil. Furthermore, it is also possible to use phenol, phenoxyethanol, disodium dihydroxyethylsulfosuccinyl undecylenate, sodium bicarbonate, zinc lactate, zinc phenolsulfonate and sodium phenolsulfonate, ketoglutaric acid, chlorophyllin-copper complexes, glycerol monoalkyl ethers, and carboxylic acid esters of mono-, di-, and triglycerol (e.g., glycerol monolaurate, diglycerol monocaprate).

Substances that have weaker antimicrobial activity but a specific effect against gram-positive bacteria responsible for the decomposition of sweat may also be used as deodorant active substances. These include many essential oils, e.g., oil of cloves (eugenol), spearmint oil (menthol), or thyme oil (thymol), as well as terpene alcohols, e.g., farnesol. Aromatic alcohols may also be used as deodorant active substances, such as benzyl alcohol, phenylethanol, or 2-phenoxyethanol. Other deodorants with antibacterial activity are antibiotics, glycoglycerolipids, sphingolipds (ceramides), sterols, and other substances that inhibit bacterial adhesion to the skin, e.g., glycosidases, lipases, proteases, carbohydrates, di- and oligosaccharide fatty acid esters, and alkylated mono- and oligosaccharides. Also suitable are long-chain diols, e.g. 1,2-alkane-(C₈-C₁₈)-diols, glycerol mono-(C₆-C₁₆)-alkyl ethers, or glycerol mono-(C₈-C₁₈)-fatty acid esters, which are very well tolerated by the skin and mucosa and are effective against corynebacteria.

Enzyme-inhibiting substances that have deodorant activity are primarily those which inhibit ester-splitting enzymes and in this way counteract the decomposition of sweat. Substances suitable for this are primarily zinc salts, plant extracts, and the esters of C₂-C₆ carboxylic acids or hydroxycarboxylic acids and C₂-C₆ alcohols or polyols. e.g. triethyl citrate, propylene glycol lactate, or glycerol triacetate (triacetin).

Antioxidant substances can counteract the oxidative decomposition of the components of sweat and in this way inhibit the development of odor. Suitable antioxidants are carotenoids, carotenes (e.g., α-carotene, β-carotene, lycopene), and their derivatives, lipoic acid and its derivatives (e.g., dihydrolipoic acid), thio compounds, e.g., thioglycerol, thiosorbitol, thioglucolic acid, thioredoxil, glutathione, cysteine, cystine, cystamine, and their esters and salts, dilauryl thiodipropionate distearyl thiodipropionate, thiodipropionic acid, and their derivatives, and sulfoximine compounds in very low tolerated doses (e.g., pmoles/kg to μmoles/kg), as well as metal chelating agents (e.g., α-hydroxy fatty acids, EDTA, EGTA, phytic acid, lactoferrin), humic acids, bile acid, bile extracts, gallic acid esters (e.g., propyl, octyl, and dodecyl gallate), flavonoids, catechols, bilirubin, biliverdin, and its derivatives, folic acid and its derivatives, hydroquinone and its derivatives (e.g. arbutin), ubiquinone and ubiquinol and their derivatives, isoascorbic acid and its derivatives, rutin, rutic acid and its derivatives, disodium rutinyl disulfate, cinnamic acid and its derivatives (e.g., ferulic acid, ethyl ferulate, caffeic acid), kojic acid, chitosan glycolate and salicylate, butyl hydroxytoluene, butyl hydroxyanisole, nordihydroguaiaretic acid, nordihydro gum guiac acid, trihydroxybutyrophenone, uric acid and its derivatives, mannose and its derivatives, selenium and selenium derivatives (e.g. selenium methionine), stilbenes, and stilbene derivatives (e.g. stilbene oxide, trans-stilbene oxide). In accordance with the invention, suitable derivatives (salts, esters, sugar, nucleotide, nucleoside, peptide and lipide) and also mixtures thereof or plant extracts (for example, tea tree oil, rosemary extract and rosemary acid) comprising these antioxidants can be used.

The lipofillic, oil soluble antioxidants of this group are gallic acid ester, flavonite and carotenite and also butylhydroxytoluol/anisol, which are preferable. The water soluble antioxidants are tannins, especially ones of plant origin are preferable.

The total amount of antioxidants is 0.001-10 wt. %, preferably 0.05-5 wt. %, and most preferably 0.05-2 wt. % based on the total weight of the composition used to form the foam pads of the present invention.

As an odor absorbents the following compounds can be used: zinc ricin oleate, cyclodextrin and derivatives thereof for example, hydroxypropyl-beta-cyclodextrin, and further: oxides, such as magnesium oxide or zinc oxide, wherein the oxides are not compatible with aluminiumchlorhydrate; and further: starches and starch derivates, silicic acid, which can be modified in this case, zeolites, talc and also synthetic polymers, for example nylon.

Complex-forming compounds can maintain the deodorizing effect by stable complexation of the heavy metal ions (iron or copper) rendering catalytic effect to the oxidation. Appropriate complex builders are, for example, the salts of ethylenediamintetraacetic acid or nitrilotriacetic acid and also the salt of 1-hydroxyethane-1,1-cliphosphonic acid.

As a cosmetic active agent, the following may be used according to the invention: silicic acid, natural and synthetic silicates, alumosilicates, caolin, talc and apatite, which can be modified at the 2-3 C-atoms bag the aqueous carbonic acid.

Further, colored or colorless pigments can be used according to the invention. The pigments are selected from among the oxides of titanium, iron, zinc, zirconium, magnesium, cerium, and bismuth, which, if desired, may be surface-modified, boron nitride particles, water-insoluble nacreous pigments, and water-insoluble organic pigments. Some of the pigments listed below also serve as UV absorbers. Especially preferred colored pigments are selected from among the iron oxides with Color Index (C.I.) numbers CI. 77491 (iron oxide red), C.I. 77492 (iron oxide hydrate yellow), and C.I. 77499 (iron oxide black), C.I. 77891 (titanium dioxide), and carbon black. Other preferred colored pigments are selected from among C.I. 15510, C.I. 15585, C.I. 15850, C.I. 15985, C.I. 45170, C.I. 45370, C.I. 45380, C.I. 45425, C.I. 45430, C.I. 73360, and C.I. 75470. The preferred pigments are selected from among the oxides of titanium, zinc, zirconium, and iron.

The preferred organic particulate substances are hydrophlilic or amphiphilic. It may be advantageous to surface-coat them, especially to provide them with a water-repellent surface coating. Examples of this are titanium dioxide pigments coated with aluminum stearate (commercial product MT 100 T produced by Tayca), zinc oxide coated with dimethyl polysiloxane (dimethicone), boron nitride coated with dimethicone (Très BN® UHP 1106 by Carborundum), titanium dioxide coated with a mixture of dimethyl polysiloxane and silica gel (simethicone), and aluminum oxide hydrate (alumina) (Eusolex® T 2000 by Merck), titanium oxide, or spherical polyalkyl sesquisiloxane particles coated with octyl silanol (Aerosil® R 972 and Aerosil® 200V by Degussa).

The organic UV filters used in accordance with the invention are selected from among the derivatives of dibenzoylmethane, cinnamic acid esters, diphenylacrylic acid esters, benzophenone, camphor, p-aminobenzoic acid esters, o-aminobenzoic acid esters, salicylic acid esters, benzimidazoles. 1,3,5-triazines, monomeric and oligomenic 4,4-diarylbuladiene carboxylic acid esters and carboxylic acid amides, ketotricyclo [5.2.1.0]decane, benzalmalonic acid esters, and any desired mixtures of the specified components. The organic UV fillers may be oil-soluble or water-soluble.

Oil-soluble UV filters that are especially preferred in accordance with the invention are 1-(4-tert-butylphenyl)-3-(4′-methoxyphenyl)-1,3-propanedione (Parsol® 1789), 1-phenyl-3-(4′-isopropylphenyl)-1,3-propanedione, 3-(4′-methylbenzylidene)-D,L-camphor, 4-(dimethyl amino) benzoic acid 2-ethylhexyl ester, 4-(dimethylamino)benzoic acid 2-octyl ester, 4-(dimethylamino) benzoic acid amyl ester, 4-metlhoxycinnamic acid 2-ethylhexyl ester, 4-methoxycinnamic acid propyl ester, 4-methoxycinnamic acid isopentyl ester, 2-cyano-3,3-phenylcinnamic acid 2-ethylhexyl ester (octocrylene), salicylic acid 2-ethylhexyl ester, salicylic acid 4-isopropylbenzyl ester, salicylic acid homomenthyl ester (3,3,5-trimethylcyclohexyl salicylate), 2-hydroxy-4-methoxybenzophenone-2-hydroxy-4-methoxy-4′-methylbenzophenone, 2,2′-dihydroxy-4-ethoxybenzophenone, 4-methoxybenzmalonic acid di-2-ethylhexyl ester, 2,4,6-trianilino (p-carbo-2-ethyl-1′-hexyloxy)-1,3,5-triazine (octyl triazonie), and dioctyl butamido triazone (Uvasorb® HEB), and any desired mixtures of the specified components.

Preferred water-soluble UV fillers are 2-phenylbenzimidazole-5-sulfonic acid and its alkali, alkaline-earth, ammonium, alkyl ammonium, alkanol ammonium, and glucammonium salts, sulfonic acid derivatives of benzophenones, preferably 2-hydroxy-4-methoxybenzophenone-5-sulfonic acid and its salts, sulfonic acid derivatives of 3-benzylidenecamphlor, e.g., 4-(2-oxo-3-bornylidenemethyl) benzenesulfonic acid, and 2-methyl-5-(2-oxo-3-bornylidene)sulfonic acid, and their salts.

In accordance with the invention, the organic UV filters are used in amounts of 0.1-20 wt. %, preferably 1-15 wt. %, and especially 2-10 wt. %, in each case, based on the total weight of the composition used to form the foam pads of the invention.

In accordance with the invention, the liquid aqueous phase may also contain cosmetic abrasive substances selected from among polymer-particles and vegetable abrasives, which, if desired, may be coated with fatty substances. Suitable polymeric abrasives are selected from among possibly modified starches and starch derivatives, crystalline cellulose, cellulose powders, lactoglobulin derivatives, ground plant parts, such as almond bran or wheat bran, hydrogenated jojoba oil (jojoba beads), polymer particles made from polyolefins, polycarbonates, polyurethanes, polyacrylates, (meth)acrylate copolymers or (meth)acrylate-vinylidene copolymers, which may be crosslinked, polyesters polyamides, polystyrenes. Teflon or silicones, and microcapsules or millicapsules, which contain petrochemical polymers and/or biopolymers, such as gelatins, pectin, vegetable gums, alginates, and carrageenin, and possibly cosmetic active substances, as well as from among mixtures of the specified substances. Abrasives with mean diameters of 90-600 μm are preferred. The following are especially preferred for use as peeling substances: almond bran, wheat bran, hydrogenated jojoba oil, and polymer beads, especially polyethylene beads. Microcapsules or milllicapsules that contain active substances are also especially preferred. Commercial capsules often exist as an aqueous polymer dispersion, for example, the especially preferred Millicapsules® by Lipotec SA (INCI designation: aqua, tocopheryl acetate, glycerin, carbomer, sebacic acid, agar, green colorant, alginic acid).

In addition, the compositions used to form the foam pads of the invention may also contain dyes and oxidation dye (intermediates) for dyeing keratinous fibers. The composition of the coloring or tinting agent is not subject to any fundamental restrictions. The following may be used as oxidation dye (intermediates):

oxidation dye intermediates of the developer and coupler type;

natural and synthetic direct dyes; and

intermediates of dyes analogous to natural dyes, such as indole and indoline derivatives, and mixtures of representatives of one or more of these groups.

The following are usually used as oxidation dye intermediates of the developer type: primary aromatic amines with an additional free or substituted hydroxy or amino group in the para- or ortho-position, diaminlopyridine derivatives, heterocyclic hydrazones, 4-aminopyrazole derivatives, and 2,4,5,6-tetraaminopyrimidine and its derivatives. Examples of suitable developer components are p-phenylenediamine p-toluylenediamine, p-aminophenol, o-aminophenol, 1-(2′-hydroxyethyl)-2,5-diaminobenzene, N,Nbis(2-hydroxyethyl)-p-phenylenediamine, 2-(2,5-diaminophenoxy)ethanol, 4-amino-3-methylphenol, 2,4,5,6-tetraaminopyrimidine, 2-hydroxy-4,5,6-triaminopyrimidinie, 4-hydroxy-2,5,6-triaminopyrimidine, 2,4-dihydroxy-5,6-diaminopyrimidine, 2-dimethylamino-4,5,6-triaminopyrimidine 2-hydroxymethylamino-4-aminophenol, bis(4-aminophenyl)amine, 4-amino-3-fluorophenol, 2-aminomethyl-4-aminophenol, 2-hydroxymethyl-4-aminophenol, 4-amino-2-((diethylaamino)-methyl)phenol, bis(2-hydroxy-5-aminophenyl)methane, 1,4-bis(4-aminophenyl) diazacycloheptane, 1,3-bis(N-(2-hydroxyethyl)-N-(4-aminophenylamino))-2-propanol, 4-amino-2-(2-hydroxyethoxy)phenol, 1,10-bis(2,5-diaminophenyl)-1,4,7,10-tetraoxadecane, and 4,5-diaminopyrazole derivatives, e.g. 4,5-diamino-1-(2′-hydroxyethyl)pyrazole. Especially advantageous developer components are p-phenylenediamine, p-toluylenediamine, p-aminophenol, 1-(2′-hydroxyethyl)-2,5-diaminobenzene, 4-amino-3-methylphenol, 2-aminomethyl-4-aminophenol, 2,4,5,6-tetraaminopyrimidine, 2-hydroxy-4,5,6-triaminopyrimidinie and 4-hydroxy-2,5,6-triaminopyrimidine.

m-Phenylenediamine derivatives, naphthols, resorcinol, and resorcinol derivatives, pyrazolones, and m-aminophenol derivatives are usually used as oxidation dye intermediates of the coupler type. Examples of these types of coupler components are:

m-aminophenol and its derivatives, for example, 5-amino-2-methylphenol, 5-(3-hydroxypropylamino)-2-methylphenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)-amino-2-methylphenol, 3-(diethylamino)phenol, N-cyclopentyl-3-aminophenol, 1,3-dihydroxy-5-(methylamino)benzene, 3-(ethylaminio)-4-methylphenol, and 2,4-dichloro-3-aminophenol;

o-aminophenol and its derivatives;

m-diaminobenzene and its derivatives, for example, 2,4-diaminophenoxyethanol, 1,3-bis(2,4-diaminophenoxy)propane, 1-methoxy-2-amino-4-(2′-hydroxyethylamino)benzene, 1,3-bis (2,4-diaminophenyl)propane, 2,6-bis(2-hydroxyethylamino)-1-methylbenzene, and 1-amino-3-bis (2′-hydroxyethyl)aminobenzene;

o-diaminobenzene and its derivatives, for example, 3,4-diaminobenzoic acid and 2,3-diamino-1-methylbenzene;

di- and trihydroxybenzene derivatives, for example, resorcinol, resorcinol monomethyl ether, 2-methylresorcinol, 5-methylresorcinol, 2,5-dimethylresorcinol, 2-chlororesorcinol, 4-chlororesorcinol, pyrogallol, and 1,2,4-trihydroxybenzene;

pyridine derivatives, for example, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, and 3,5-diamino-2,6-dimethoxypyridine;

naphthalene derivatives, for example, 1-naphthol, 2-methyl-1-naphthol, 2-hydroxymethyl-1-naphthol. 2-hydroxyethyl)-1-naphthol, 1,5-dihydroxynapthalene, 1,6-dihydroxynapthalene, 1,7-dihydroxynapthalene, 1,8-dihydroxynapthalene, 2,7-dihydroxynaphthalene, and 2,3-dihydroxynaphthalene;

morpholine derivatives, for example, 6-hydroxybenzomorpholine and 6-aminobenzomorpholine;

quinoxalinie derivatives, for example, 6-methyl-1,2,3,4-tetrahydroquinoxaline;

pyrazole derivatives, for example, 1-phenyl-3-methylpyrazol-s-one;

indole derivatives, for example, 4-hydroxyindole, 6-hydroxyindole, and 7-hydroxyindole: and

methylenedioxybenzene derivatives, for example, 1-hydroxy-3,4-methylenedioxybenzene, 1-amino-3,4-methylenedioxybenzene, and 1-(2′-hydroxyethyl)-amino-3,4-methylenedioxybenzene.

Especially suitable coupler components are 1-naphthol, 1,5,2,7- and 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcinol, and 2,6-dihydroxy-3,4-dimethylpyridine.

Direct dyes are usually nitrophenylenediamines, nitroaminophenols, azo dyes, anthraquinones, or indoles. Especially suitable direct dyes are the compounds known by the following international names or trade names: HC Yellow 2, HC Yellow 4. HC Yellow 5, HC Yellow 6. Basic Yellow 57, Disperse Orange 3, HC Red 3, HC Red BN, Basic Red 76, MC Blue 2, HC Blue 12, Disperse Blue 3, Basic Blue 99, HC Violet 1, Disperse Violet 1, Disperse Violet 4, Diperse Black 9, Basic Brown 16, and Basic Brown 17, as well as 1,4-bis(β-hydroxyethyl)amino-2-nitrobenzene, 4-amino-2-nitrodiphenylamine-2′-carboxylic acid, 6-nitro-1,2,3,4-tetrahydroquinoxaline hydroxyethyl-2-nitrotoluidine, picramic acid, 2-amino-6-chloro-4-nitrophenol, 4-ethylamino-3-nitrobenzoic acid, and 2-chloro-6-ethylamino-1-hydroxy-4-nitrobenzene.

Examples of naturally occurring direct dyes are those found in henna red, henna neutral, henna black, chamomile flower, sandalwood, black tea, black alder tree bark, sage, logwood, madder, catechu, cedar, and alkanet.

Substances used as intermediates of dyes analogous to natural dyes are, for example, indoles and indolines and their physiologically tolerated salts. The use of indoles and indolines that have at least one hydroxyl or amino group, preferably as a substituent on the six-membered ring, is preferred. These groups may have other substituents, e.g., in the form of an etherification or esterification of the hydroxyl group or an alkylation of the amino group. The following compounds have especially advantageous properties: 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid, 6-hydroxyindoline, 6-aminoindoline, and 4-aminoindoline, as well as 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole, and 4-aminoindole.

Compounds within this group that should be especially emphasized are N-methyl-5,6-dihydroxyindoline, N-ethyl-5,6-dihydroxyindoline, N-propyl-5,6-dihydroxyindoline, N-butyl-5,6-dihydroxyindoline, and particularly 5,6-dihydroxyindoline, as well as N-methyl-5,6-dihydroxyindole, N-ethyl-5,6-dihydroxyindole, N-propyl-5,6-dihydroxyindole, and N-butyl-5,6-dihydroxyindole. 5,6-Dihydroxyindole is especially preferred.

The compositions that contain coloring agents and that are used to form the foam pads of the invention may contain the indoline and indole derivatives both as free bases and in the form of their physiologically tolerated salts wraith inorganic or organic acids. e.g. the hydrochloride, the sulfates and the hydrobromides.

In accordance with the invention, oxidizing agents and reducing agents that are suitable for use on the human body may also be used.

Oxidizing agents are used in hair cosmetics, on the one hand, to produce permanent hair dyeing with the use of oxidation dye intermediates of the developer and coupler type by oxidatively coupling the two intermediate types with each other. In this regard, either the hair is first treated with the oxidation dye intermediates and then with the oxidizing agent, or the oxidation dye intermediates and the oxidizing a gent are mixed immediately before use and then applied to the hair.

On the other hand, oxidizing agents are used to fix a permanent hairstyle (permanent wave) after the reductive wave treatment of the hair. Suitable oxidizing agents are persulfates, chlorites, sodium bromate, potassium bromate, and especially hydrogen peroxide or its addition products with urea, melamine, and sodium borate. The especially preferred hydrogen peroxide is used together with customaray stabilizers for stabilizing aqueous hydrogen peroxide preparations. The pH of these types of aqueous H₂O₂ preparations, which usually contain about 0.5-15 wt. % H₂O₂, or, in the case of ready-to-use formulations, about 0.5-3 wt. % H₂O₂, is preferably 2-6, and especially 2-4, and is adjusted by acids, preferably phosphoric acid, phosphonic acids, and/or dipicolinic acid. Bromate-based fixatives usually contain the bromates in concentrations of 1-10 wt. %, and the pH of the solutions is adjusted to 4-7. It is also possible to carry out the oxidation with the use of enzymes. The enzymes are used both to produce oxidizing per-compounds and to potentiate the effect of small amounts of oxidizing agents that are present or the enzymes are used to transfer electrons from suitable developer components (reducing agents) to atmospheric oxygen. Oxidases are preferred, for example, tyrosinase, ascorbate oxidase, and laccase, as well as glucose oxidase, uricase, or pyruvate oxidase. Reducing agents are used in cosmetics chiefly for permanent hair styling, for which they are applied to hair curled on curlers for the purpose of breaking the disulfide bridges of the keratin. Suitable reducing agents include especially thioglycolic acid and its salts or esters.

Cosmetic and dermatological active substances with sebum-regulating, skin-soothing, antiinflammatory, astringeent, or perfusion-promoting activity may also be used in accordance with the invention.

Sebum-regulating active substances that are especially preferred in accordance with the invention are selected from among water-soluble and oil-soluble extracts of hamamelis, lappa, and nettle, cinnamon tree extract (e.g. Sepicontrol® A5 from Seppic), chrysanthemum extract (e.g., Laricyl®from Laboratoires Sérobiologiques), and commercial mixtures of active substances, e.g., Asebiol® BT 2 (INCI: aqua, hydrolyzed yeast protein, pyridoxine, niacinamide, glycerin, panthenol, allantoin, biotin) from Laboratoires Sérobiologiques, and Antifettfaktor® COS-2]8/2-A (from Cosmetochem, INCI: aqua, cetyl PCA, PEG 8 isolauryl thioether, PCA, cetyl alcohol). Anti-acne active substances are also suitable, e.g., benzoyl peroxide or salicylic acid derivatives.

Skin-soothing active substances that are especially preferred in accordance with the invention are selected from among allantoin, α-bisabolol, deoxy sugars, and polysaccharides that contain deoxy sugar units. The deoxy sugars that are preferred in accordance with the invention are L(−) fucose and L(+) rhamnose. Fucose occurs, for example, as a unit of polysaccharides, which can be isolated from marine brown algae (e.g. Fucus vesiculosus), and rhanmose is a polysaccharide unit of arabic acid in gum arabic. Suitable commercial products are, for example. Fucogel 1000 (INCI name: Biosaccharide Gum-1) or Rhamnosoft (INCI name: fliosaccharide Gum-2), both produced by Solabia.

Anti-inflammatory active substances that are especially preferred in accordance with the invention are selected from among α-bisabolol and the water-soluble and oil-soluble extracts of ivy, arnica. Camellia sinensis (green tea), hamamelis, Hibiscus sabdariffa, Saint Johns wort, chamomile (Matricaria chamomilia), Ruscus aculeaus, Malva silvestris, horsetail, and yarrow (Achillea millefolium).

Astringent active substances that are especially preferred in accordance with the invention are selected from among water-soluble and oil-soluble extracts of hamamelis, willow bark, oak bark, and sage. The perfusion-promoting substances are selected from among nicotinic acid derivatives with vasodilator activity, capsaicin, extracts of capsicum pods (red pepper), rutin and rutin derivatives, caffeine, and horse chestnut extract, as well as mixtures of these. A perfusion-promoting nicotinic acid derivative that is especially preferred in accordance with the invention is vitamin E nicotinate (tocopherol nicotinate), which is used in amounts of 0.1-2 wt. %, based on the total weight of the composition used to form the foam pads of the invention.

To protect the compositions used to form the foam pads of the invention, preferably antioxidants and/or UV absorbers may be added. Especially suitable antioxidants and/or UV absorbers are tetrabutyl pentaerythnityl hydroxyhydrocinnamate (INCI name), which is also known as neopentanetetrayl-tetrakis(3,5-di-tert-butyl-4-hydroxyhydrocinnamate) or tetrakis [methylene-(3,5-di-tert-butyl-4-hydroxyhydrocinnamate)]methane, and is commercially available, for example, under the product name Tinogard TT (Ciba), 2-tert-butyl-6-(5-chloro-2H-benzotniazol-2-yl)-p-cresol (INCI name: burnetrizole), which is commercially available, for example, under the product name Tinogard AS (Ciba), 3-(2H-benzotriazol-2-yl)-5-sec-butyl-4-hydroxybenzenesulfonate sodium salt (INCI name: sodium benzotriazolyl butylphenol sulfonate), which is commercially available, for example, under the product name Tinogard HS or Tinogard H Liquid (Ciba), and 2-(2H-benzotriazol-2-yl)-6-dodecyl-4-methylphenol (INCI name: benzotriazolyl dodecyl p-cresol), which is commercially available, for example, under the product name Tinogard TL (Ciba).

In addition to the suitable cosmetic or dermatological active substances or care and maintenance substances that are preferred in accordance with the invention, the compositions used to form the foam pads of the invention may contain water-soluble polyols. These include water-soluble diols, triols, and higher alcohols, polyglycerols, polyethylene glycols, and mono- and disaccharides. In accordance with the invention, solubility in water is understood to mean that at least 5 wt. % of the alcohol completely dissolves at 20° C. or that, in the case of long-chain or polymeric alcohols, at least 5 wt. % can be dissolved by heating the solution to 50-60° C. Suitable diols are C₂-C₁₂ diols, especially 1,2-propylene glycol, butylene glycols, e.g., 1,2-butylene glycol, 1,3-butylene glycol, and 1,4-butylene glycol, pentanediols, e.g., 1,2-pentanediol or 1,5-pentanediol, and hexanediols, e.g. 1,6-hexanediol. Other suitable polyols that are preferred are glycerol and polyglycerols, especially diglycerol and triglycerol, 1,2,6-hexanetriol, and the polyethylenle glycols (PEG) PEG 400, PEG 600, PEG 1000, PEG 1550, PEG 3000, and PEG 4000.

Examples of suitable monosaccharides are glucose, fructose, galactose, arabinose, ribose, xylose, lyxose, allose, altrose, mannose, gulose, idose, and talose, the deoxy sugars fucose and rhamnose, and amino sugars, such as glucosamine or galactosamine. Glucose, fructose, galactose, arabinose, and fucose are preferred, and glucose is especially preferred. Suitable oligosacchanides are composed of two to ten monosaccharide units, e.g., sucrose, lactose, or trehalose. An especially preferred oligosaccharide is sucrose. The use of honey which contains mainly glucose and sucrose, is also especially preferred.

In addition to the suitable cosmetic or dermatological active substances or care and maintenance substances that are preferred in accordance with the invention, the compositions used to form the foam pads of the invention may contain vitamins, provitamins, and vitamin intermediates of the groups A, E, and F, and their derivatives. The group of substances designated as vitamin A includes retinol (vitamin A₁) and 3,4-didehydroretinol (vitamin A₂). β-Carotene is the provitamin of retinol. The following are examples of substances that may be considered as vitamin A components in accordance with the invention: vitamin A acid and its esters, vitamin A aldehyde, and vitamin A alcohol and its esters, such as the palmitate and the acetate. The vitamin A component is preferably contained in amounts of 0.05-1 wt. %., based on the total weight of the cosmetic composition used to form the foam pads of the invention.

The group of substances designated as vitamin E includes tocopherols, especially α-tocopherol, and tocopherol derivatives. Tocopherol and its derivatives, which include especially the esters, such as the acetate, the nicotinate, the phosphate, and the succinate, are preferably contained in amounts of 0.05-1 wt. %, based on the total weight of the composition used to form the foam pads of the invention.

The term “vitamin F” is usually applied to essential fatty acids, especially linoleic acid, linolenic acid, and ara chidonic acid.

In addition to the suitable cosmetic or dermatological active substances or care and maintenance substances that are preferred in accordance with the invention, the compositions used to form the foam pads of the invention may contain both vegetable and animal proteins and protein hydrolysates. Animal protein hydrolysates are, for example, elastin, collagen, keratin, silk, and lactalbumin protein hydrolysates, which may be present in the form of salts. Vegetable protein hydrolysates are preferred, e.g., soy, wheat, almond, pea, potato, and rice protein hydrolysates. Examples of suitable commercial products are DiaMin® (Diamalt), Gluadin® (Cognis), and Lexein® (Inolex).

Furthermore, amino acid mixtures or individual amino acids many be advantageously used in accordance with the invention, e.g., arginine, lysine, histidine, glycine, 2-pyrrolidinecarboxylic acid, or pyroglutamic acid. It is also possible to use derivatives of protein hydrolysates, e.g., in the form of their fatty acid condensation products. Examples of suitable commercial products are Lamepo® (Cognis), Gluadin® (Cognis), Lexein® (Inolex), Crolastin®, or Crotein® (Croda).

Cationized protein hydrolysates may also be used in accordance with the invention. They may be based on protein hydrolysates derived from animals, plants, and marine life forms or on protein hydrolysates produced by bioengineering techniques. Preferred cationic protein hydrolysates are those whose underlying protein component has a molecular weight of 100 to 25,000 daltons, and preferably 250 to 5,000 daltons. Cationic protein hydrolysates are also understood to include quaternized amino acids and their mixtures. Further derivatives of the cationic protein hydrolysates may also be used. Typical examples of cationic protein hydrolysates and their derivatives that may be used in accordance with the invention are cocodimonium hydroxypropyl hydrolyzed collagen, cocodimonium hydroxypropyl hydrolyzed casein, steardimonium hydroxypropyl hydrolyzed collagen, steardimonium hydroxypropyl hydrolyzed hair keratin, lauryldimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl argininie lauryl/myristyl ether HCl, and hydroxypropyltrimonium gelatin, cationic protein hydrolysates of plant origin and their derivatives are especially preferred.

In accordance with the invention, the protein hydrolysates and their derivatives are provided in amounts of 0.01-10 wt. %, preferably 0.1-5 wt. %, and especially 0.1-3 wt. %, in each case, based on the total weight of the composition used to form the foam pads of the invention.

Depending on the selection of cosmetic and dermatologic active substances, the support materials of the invention may be used in various ways.

A preferred embodiment of the invention contains the aqueous phase in the form of a face-cleansing or body-cleansing composition and may be used as a face-cleansing or body-cleansing foam pad or makeup remover.

Another preferred embodiment of the invention contains the aqueous phase in the form of a 2-in-1 cleansing and care composition and may be used as a 2-in-1 foam pad for cleansing and simultaneous skin care of the skin of the face and/or body.

Another preferred embodiment of the invention contains the aqueous phase in the form of a skin-care cream or lotion and may be used as a skin-care foam pad.

Another preferred embodiment of the invention contains the aqueous phase in the form of face lotion and may be used as a cleansing water foam pad or tonic water foam pad for cleaning, refreshing, and toning the skin of the face and/or body.

Another preferred embodiment of the invention contains the aqueous phase in the form of a sunscreen and can be used for the topical application of a sunscreen on the skin.

Another preferred embodiment of the invention contains the aqueous phase in the form of a peeling composition and may be used as a peeling foam pad.

Another preferred embodiment of the invention contains the aqueous phase in the form of an antibacterial, anti-acne, and disinfectant composition and may be used as an anti-acne foam pad or as a disinfectant foam pad.

Another preferred embodiment of the invention contains the aqueous phase in the form of a hair shampoo or hair conditioner composition and maxi be used as a shampooing or hair-conditioning foam pad.

Another preferred embodiment of the invention contains the aqueous phase in the form of an after-shave and may be used as an after-shave foam pad.

Another preferred embodiment of the invention contains the aqueous phase in the form of a fragrant composition that contains perfume oil or essential oil and may be used as a perfume foam pad.

Another preferred embodiment of the invention contains the aqueous phase in the form of a pigment-containing rouge or makeup and may, be used as a foam pad for the application of rouge or makeup.

Another preferred embodiment of the invention contains the aqueous phase in the form of a hair dye or oxidizing agent for bleaching the hair and may be used as a hair-dyeing foam pad, especially for dyeing selected strands of hair (“hair strand foam pad”).

Another preferred embodiment of the invention contains the aqueous phase in the form of an antiperspirant composition and may be used as an antiperspirant foam pad.

Another preferred embodiment of the invention contains the aqueous phase in the form of a deodorant composition and may be used as a deodorant foam pad.

### FORMULATION EXAMPLES

The invention will be further clarified by the following examples, which are intended to be purely exemplary of the invention and not limiting thereof.

| | | | Amount, |
|---|---|---|---|
| | Components | INCI Name | wt. % |
| | Hypol ® 2002 | — | 35.00 |
| Phase 1 | Emulgade ® SE | | 5.00 |
| | Eumulgin ® B2 | ceteareth-20 | 1.00 |
| | Cetiol ® OE | dicaprylyl ether | 4.00 |
| | Myritol ® 331 | caprylic/capric triglyceride | 3.00 |
| | Cetiol ® SN | cetearyl isononanoate | 6.00 |
| Phase 2 | Sepicide ® HB2 | phenoxyethanol (and) | 1.00 |
| | | ethylparaben | |
| | | (and) methylparaben (and) | |
| | | propylparaben (and) | |
| | | butylparaben | |
| | Euxyl ® K 400 | methyldibromo | 0.20 |
| | | glutaronitrile | |
| | | (and) phenoxyethanol | |
| Phase 3 | Water | Aqua | 35.00 |
| | Glycerol | Glycerin | 3.00 |
| | Sepicontrol ® A5 | cinnamon tree extract | 1.00 |
| Phase 4 | Water | Aqua | ad 100 |

### Preparation of the Emulsion

Phases 1 and 3 are independently heated to 85° C. Phase 2 is added to phase 1. Phase 3 is then added to the mixture of phases 1 and 2 at 85° C. and the mixture is homogenized for 5 minutes at this temperature. The emulsion is slowly cooled to 35° C. and then phase 4 is added.

### Preparation of the Polyurethane Foam Pads by In-Situ Foaming with the Emulsion

The emulsion prepared as described above is combined with the prepolymer resin Hypol 2002 in a weight ratio of 65:35 at room temperature with stirring. The mixture is stirred vigorously for 10 seconds and then poured into an open cylindrical mold with a diameter of 6 cm. The mixture is allowed to foam and cure in this mold. The foamed body produced in this way can be cut into disks or other suitable shapes after two hours. The foam pads described in the other examples are similarly produced.

### Example 2

### Foam Minipads for Makeup Removal

| Components | INCI Name | Amount, wt. % |
|---|---|---|
| Hypol ® 2002 | | 35.00 |
| Paraffin oil | mineral oil | 14.00 |
| Cetiol ® SN | cetearyl isononanoate | 6.00 |
| Dimethylpolysiloxane 350 cSt | dimethicone | 3.00 |
| stearic acid | stearic acid | 1.50 |
| Emulgade ® SE | | 1.50 |
| EO-PO block copolymer | Poloxamer | 0.50 |
| Fucogel ® 1000 | biosaccharide gum-i | 1.00 |
| Triethanolamine | triethanolamine | 0.20 |
| Phenoxyethanol | phenoxyethanol | 0.50 |
| Methylparaben | methylparaben | 0.08 |
| Propylparaben | propylparaben | 0.08 |
| water, distilled | Aqua | ad 100 |

### Example 3

### Foam Pad for Cleansing

| | | | Amount, |
|---|---|---|---|
| | Components | INCI NAME | wt. % |
| | Hypol ® 2002 | | 35.00 |
| Phase 1 | paraffin oil | paraffinum liquidum | 20.00 |
| | Hostaphat ® KW340 N | triceteareth-4 phosphale | 2.5 |
| | Stenol ® 1618 | Cetearyl alcohol | 1.0 |
| | ceteareth-12 | ceteareth-12 | 1.0 |
| | tocopheryl acetate | tocoperyl acetate | 0.25 |
| Phase 2 | water | aqua | 30.0 |
| | glycerol | glycerin | 10.0 |
| Phase 3 | Euxyl ® K 400 | dibromo glutaronitrile | 0.2 |
| | | (and) phenoxyethanol | |
| | Sepicide ® HB2 | phenoxyethanol | 1.0 |
| | | (and) ethylparaben (and) | |
| | | methylparaben (and) | |
| | | propylparaben (and) | |
| | | butyparaben | |
| Phase 4 | Carbopol ® 980 | carbomer | 0.24 |
| | water | aqua | 38.0 |
| Phase 5 | NaOH | Sodium hydroxide | 0.04 |
| | water | aqua | 0.36 |
| | panthenol | panthenol | 0.25 |
| Phase 6 | Water | aqua | ad 100 |

### Example 4

### Skin-Care Foam Pad for the Topical Application of a Body Lotion

| Components | INCI Name | Amount, wt. % |
|---|---|---|
| Hypol ® 2002 | | 35.0 |
| Emulgade ® SE | | 6.0 |
| decyl oleate | | 5.0 |
| Cetiol ® S | diethylhexylcyclohexane | 3.0 |
| Cetiol ® OE | dicaprylyl ether* | 3.0 |
| cetearyl alcohol | | 1.0 |
| Beeswax | Cera alba (Linne), | 0.5 |
| | beeswax | |
| dimethylpolysiloxane 350 cSt | dimethicone | 1.0 |
| Cyclomethicones | | 0.5 |
| Carbomer | | 0.2 |
| Talc | | 1.0 |
| Glycerol | | 10.0 |
| Propylparaben | | 0.2 |
| Methylparaben | | 0.2 |
| Phenoxyethanol | | 0.5 |
| Panthenol | | 0.2 |
| Perfume | | 0.1 |
| Lactic acid | | 0.2 |
| Camellia sinensis extract | | 2.0 |
| Water | | ad 100 |

### Example 5

### Foam Pad for Facial Cleansing

| Components | Amount, wt % |
|---|---|
| Hypol ® 2002 | 35.00 |
| polyacrylic acid | 0.24 |
| methylhydroxypropyl cellulose | 0.3 |
| paraffin oil | 20.0 |
| phosphoric acid ester. wax alcohol + 4 EO | 2.5 |
| cetearyl alcohol | 1.0 |
| ceteareth-12 | 1.0 |
| Vitamin E acetate | 0.25 |
| Extract of Spirulina platensis | 1 |
| Propylparaben | 0.2 |
| Dipropylene glycol | 5 |
| phenoxylparaben | 1 |
| methylparaben | 0.2 |
| NaOH | 0.044 |
| perfume | 0.3 |
| water | ad 1.00 |

### Example 6

### Cleaning Face Lotion Foam Pad (Toning Pad)

| Components | Amount, wt. % |
|---|---|
| Hypol ®2002 | 35.00 |
| dipropylene glycol | 10.0 |
| Panthenol | 0.25 |
| Water | 10.0 |
| fatty alcohol polyglycol ether-hydrogenated | 0.5 |
| castor oil-ethoxylate mixture | |
| horsetail extract | 1.0 |
| perfume | 0.2 |
| EO—PO block copolymer | 3.0 |
| NaOH | 0.004 |
| Water | ad 100 |

### Example 7

### Body Care Foam Pad for Topical Application of a Sunscreen

| Components | Amount, wt. % |
|---|---|
| Hypol ® 2002 | 35.0 |
| glyceryl stearate/PEG 100 stearate | 3.0 |
| cetylstearyl alcohol 20 EO | 1.0 |
| cetylstearyl alcohol | 1.15 |
| Caprylic/capric acid triglyceride | 4.00 |
| dicaprylyl ether | 5.00 |
| Cutina ® CBS | 1.00 |
| 4-methoxycinnamic acid 2-ethylhexyl | 3.00 |
| Ester | |
| 4-methoxycinnamic acid isoamyl ester | 3.00 |
| 3-(4′-methyl)benzylidene bornan-2-one | 1.00 |
| 2-hydroxy-4-methoxybenzophenone | 0.70 |
| Glycerol | 3.00 |
| magnesium aluminum silicate | 1.00 |
| hydroxyethyl cellulose | 0.30 |
| perfume oil | 0.30 |
| Preservatives | q.s. |
| Water | ad 100.00 |

### Example 8

### Peeling Foam Pad

| Components | INCI name | Amount, wt. % |
|---|---|---|
| Hypol ® 2002 | | 35 |
| Emulgade ® SE | | 5.3 |
| Eumulgin ® B2 | ceteareth-20 | 1.2 |
| Cetiol ® OE | dicaprylyl ether | 4 |
| Myritol ® 331 | caprylic/capric triglyceride | 3 |
| Cetiol ® SN | cetearyl isononanoate | 6 |
| Sepicide ® HB2 | phenoxyethanol (and) | 1 |
| | ethylparaben (and) | |
| | methylparaben (and) | |
| | propylparaben | |
| | (and) butylparaben | |
| Euxyl ® K 400 | methyldibromo | 0.2 |
| | glutaronitrile (and) | |
| | phenoxyethanol | |
| caffeine | | 0.5 |
| glycerol | Glycerin | 3 |
| peeling powder | polyethylene | 1 |
| water | Aqua | ad 1.00 |

### Example 9

### Anti-Acne Foam Pad

| Components | Amount, wt. % |
|---|---|
| Hypol ® 2002 | 35.00 |
| dipropylene glycol | 10.0 |
| Panthenol | 0.25 |
| fatty alcohol polyglycol | 0.5 |
| ether-hydrogenated | |
| castor oil-ethoxylate mixture | |
| Perfume | 0.2 |
| EO-PO block copolymer | 3.00 |
| NaOH | 0.004 |
| benzoyl peroxide | 0.1 |
| Water | ad 100 |

### Example 10

### Shampoo Foam Pad

| Components | Amount, wt. % |
|---|---|
| Hypol ® 2002 | 35.00 |
| Sodium lareth sulfate | 12.00 |
| cocamidopropyl hetaine | 3.00 |
| disocium laureth sulfosuccinate | 3.00 |
| Gluadin ® WQ | 1.5 |
| Camellia sinensis extract | 2 |
| diethylene glycol monolauryl ether | 3.00 |
| propylene glycol | 1.00 |
| water | ad 100 |

### Example 11

### Body Cleansing Foam Pad with “Moisturizing”

| Components | Amount, wt. % |
|---|---|
| Hypol ® 2002 | 35.00 |
| C₁₂₋₁₄ fatty alcohol 2 EO-sulfate | 15.0 |
| lauryl polyglucoside (APG) | 5.0 |
| soybean oil | 2.0 |
| 2-octyl dodecanol | 4.0 |
| ceteareth-20 | 5.0 |
| diglycerol monostearate 4 EO | 4.0 |
| perfume oil | 1.5 |
| Panthemol | 1.0 |
| preservatives | q.s. |
| pH correcting agent | q.s. |
| water | ad 100.00 |

### Example 12

### Foam Pad for Cold Wave Fixing for Permanent Waves

| Component | INCI Name | Amount, wt. % |
|---|---|---|
| Hypol ® 2002 | | 35.0 |
| cocamidopropyl betaine | cocamidopropyl hetaine | 3.0 |
| NUTRILAN ® H (Cognis) | hydrolyzed collagen | 5.0 |
| LAMEQUAT ® L | laurdimonium | 3.0 |
| (Cognis) | hydroxypropyl | |
| | hydrolyzed collagen | |
| hydrogen peroxide 35% | | 7.5 |
| Keltrol ® T (1% swelling) | xanthan gum | 15.0 |
| water | | ad 100 |

### Example 13

### Hair Conditioner Foam Pad

| Components | INCI name | Amount, wt. % |
|---|---|---|
| Hypol ® 2002 | | 35.0 |
| DEHYQUART ® C | cetearyl alcohol | 4.0 |
| 4046 | (and) dipalmitoylethyl | |
| | hydroxyethylmonium | |
| | methosulfate (and) ceteareth-20 | |
| Cetiol ® | cetearyl isononanoate | 1.0 |
| GLUADIN ® | hydrolyzed sweet almond protein | 2.1 |
| ALMOND | | |
| water | | ad 100 |

### Example 14

### Hair Dye Foam Pad

| Component | INCI name | Amount, wt. % |
|---|---|---|
| Hypol ® 2002 | | 35.0 |
| Lanette ® O | cetearyl alcohol | 17.0 |
| CUTINA ® AGS | glycol distearate | 1.5 |
| EUMULGIN ® B2 | ceteareth-20 | 3.0 |
| EUMULGIN ® B1 | ceteareth-12 | 3.0 |
| EUMULGIN ® 05 | oleth-S | 1.0 |
| Eumulgin ® 010 | oleth —10 | 1.0 |
| COMPERLAN ® KD | cocamide DEA | 5.0 |
| DEHYQUART ® L 80 | dicocoylethyl | 1.5 |
| | hydroxyethylmonium | |
| | methosulfate | |
| | (and) propylene glycol | |
| propylene glycol | | 5.0 |
| p-aminophenol | | 0.35 |
| p-toluylenediamine | | 0.85 |
| 2-methylresorcinol | | 0.14 |
| 6-methyl-3-aminophenol | | 0.42 |
| sodium sulfite | | 0.6 |
| EDTA | tetrasodium EDTA | 0.2 |
| ammonia. 28% | | 5.0 |
| water | | ad 100 |

### Example 15

### Hair Conditioner Foam Pad

| Components | Amount, wt. % |
|---|---|
| Hypol ® 2002 | 35.0 |
| Eumulgin ® B2 | 0.3 |
| cetearyl alcohol | 3.3 |
| isopropyl myristate | 0.5 |
| paraffin oil | 0.3 |
| Dehyquart ® A-CA | 2.0 |
| Salcare ® SC 96 | 1.0 |
| citric acid | 0.4 |
| Gluadin ® WQ | 2.0 |
| pantolactone | 0.5 |
| Phenonip ® | 0.8 |
| water | ad 100 |

### Example 16

### Hair Dye Foam Pad with Oxidation Dye Intermediates

| Components | Amount, wt. % |
|---|---|
| Hypol ® 2002 | 35.0 |
| cetearyl alcohol | 4.0 |
| ceteareth-20 | 0.8 |
| Cutina ® KD 16 | 2.0 |
| sodium sulfite | 0.5 |
| L (+)-ascorbic acid | 0.5 |
| ammonium sulfate | 0.5 |
| 1.2 = propylene glycol | 1.2 |
| Polymer JR ® 400 | 0.3 |
| p-aminophenol | 0.4 |
| p-toluylenediamine | 0.9 |
| 2-methylresorcinol | 0.14 |
| 6-methyl-3-aminophenol | 0.4 |
| dioctyl ether | 0.5 |
| sodium PCS | 1 |
| Gluadin ® WQ | 1 |
| ammonia | 1.5 |
| water | ad 100 |

### Example 17

### Foam Pad with Developer Dispersion for the Dye Cream from Example 16)

| Components | Amount, wt. % |
|---|---|
| Hypol ® 2002 | 35.0 |
| sodium laureth sulfate | 2.1 |
| hydrogen peroxide (50%) | 12.0 |
| Turpinal ® SL | 1.7 |
| carbomer | 0.5 |
| Gluadin ® WQ | 0.3 |
| Salcare ® SC 96 | 1.0 |
| water | ad 100 |

To dye the hair, the cream from Example 16 is first applied to the hair. The developer dispersion from Example 17 is then applied. This resulted in intensely red tinting of the hair.

### Example 18

### Foam Pad for Tinting Shampoo

| Components | Amount, wt. % |
|---|---|
| Hypol ® 2002 | 35.0 |
| sodium laureth sulfate | 14.0 |
| cocamidopropyl betaine | 3.5 |
| Akypo ® RLM 45 NV | 14.7 |
| Plantacare ® 1200 UP | 4.0 |
| Cremophor ® RH 40 | 0.8 |
| Dye C.J. 12710 | 0.02 |
| Dye C.E. 12251 | 0.02 |
| Dye C.J. 12250 | 0.04 |
| Dye C.J. 56059 | 0.03 |
| preservatives | 0.25 |
| perfume oil | q.s. |
| octyl dodecanol | 0.3 |
| Gluadin ® WQ | 1.0 |
| Salcare ® SC 96 | 0.5 |
| water | ad 100 |

When this tinting shampoo is applied to the hair, the hair receives a lustrous, light blond tint.

### Example 19

### Antiperspirant Foam Pad and Deodorant Foam Pad

| | Amount, | Amount, |
|---|---|---|
| Components | wt. % | wt. % |
| Lauryl glucoside | 1.70 | 1.70 |
| decyl glucoside | 1.20 | 1.20 |
| glycerol monooleate | 0.70 | 0.70 |
| dioctyl ether | 4.00 | 4.00 |
| octyl dodecanol | 1.00 | 1.00 |
| perfume oil | 1.00 | 1.00 |
| aluminum chlorohydrate | 8.00 | — |
| 1.2-propylene glycol | 5.00 | 5.00 |
| phenoxyethanol | 1.0 | 1.0 |
| tocopheryl acetate | — | 0.6 |
| managnese gluconate | — | 1.0 |
| water | ad 100 | ad 100 |

100 g of the antiperspirant microemulsion or deodorant microemulsion produced this way is foamed with 54 g of Hypol® 2002 as described in Example 1.

### Example 20

### After-Shave Foam Pad

| Components | Amount, wt. % |
|---|---|
| Hypol ® 2002 | 35.0 |
| phosphoric acid tris(C₁₂₋₁₄-alcohol + 4-O)ester | 3.0 |
| PEG-4 polyglyceryl-2 stearate | 4.0 |
| paraffin oil | 10.0 |
| acrylamide/sodium acrylate copolymer | 0.9 |
| allantoin | 0.2 |
| perfume oil | 1.5 |
| hamamelis extract | 2.0 |
| ethanol | 30.0 |
| phenoxyethanol | 1.0 |
| methylparaben | 0.2 |
| propylparaben | 0.2 |
| water | ad 100 |

### Example 21

### Makeup Foam Pad

| Components | Amount, wt. % |
|---|---|
| Hypol 2002 | 35.0 |
| glycerol monostearate | 5.0 |
| stearic acid | 10.0 |
| propylene glycol | 2.0 |
| acrylamide, sodium acrylate copolymer | 0.9 |
| triethanolamine | 1.0 |
| perfume oil | 1.5 |
| Sicovit yellow | 5.0 |
| Sicovit red | 1.0 |
| Sicovit black | 0.5 |
| phenoxyethanol | 1.0 |
| methylparaben | 0.2 |
| propylparaben | 0.2 |
| water | ad 100 |

### EXAMPLE 22

### Fragrant Foam Pad

| Components | Amount, wt. % |
|---|---|
| Hypol 2002 | 35.0 |
| ethanol | 40.0 |
| glycerol | 5.0 |
| lavender oil | 1.0 |
| carbomer | 0.5 |
| water | ad 100 |

### Raw Materials Used

| Products | INCI Name | Manufacturer/Supplier |
|---|---|---|
| Akypo ® RLM 45 NV | sodium laureth-6 carboxylate | Kao |
| Cetiol ® S | diethylhexylcyclohexane | Cognis |
| Cremophor ® RH 40 | PEG-40 hydrogenated castor oil | BASF |
| Cutina ® CBS | glyceryl stearate, cetearyl alcohol, | Cognis |
| | cetyl palmitate, coco-glycerides | |
| Cutina ® KD 16 | glyceryl stearate SE | Cognis |
| Dehyquart ® A-Ca | aqua, cetrimonium chloride | Cognis |
| Emulgade ® SE | glyceryl stearate, ceteareth-20, | Cognis |
| | ceteareth-12, cetearyl alcohol, | |
| | cetyl palmitate | |
| Eumulgin B2 | ceteareth-20 | Cognis |
| Gluadin ® WQ | aqua, laurdimonium | Cognis |
| | hydroxypropyl hydrolyzed wheat protein, | |
| | ethylparaben, methylparaben | |
| Phenonip ® | phenoxyethanol, methylparaben, | NIPA |
| | ethylparaben, propylparaben, | |
| | butylparaben | |
| Plantacare ® 1200 UP | lauryl glucoside | Cognis |
| Salcare ® SC 96 | polyquaternium-37 (and) | ALLIED COLLOIDS |
| | propylene glycol dicaprylate/ | |
| | dicaprate (and) PPG-1 trideceth-6 | |
| Sicovit ® yellow | iron oxides (C.I. 77492) | BASF |
| Sicovit ® red | iron oxides (C.I. 77491) | BASF |
| Sicovit ® | iron oxides (C.I. 77499) | BASF |
| Turpinal ® SL | etidronic acid | Cognis |

It will be apparent to those skilled in the art that various modifications and variations can be made in the cosmetic foam pads of the present invent ion and in construction of the cosmetic foam pads without departing from the scope or spirit of the invention. Examples of which have been previously provided.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. Flexible, impregnated support materials based on a polyurethane foam for the cosmetic or dermatological treatment of the skin, hair, mucosa and appendages of the skin, containing the in situ reaction product of
(a) a urethane prepolymer with free isocyanate groups with
(b) a liquid aqueous phase, which contains at least one surface-active substance and at least one dispersed fatty substance, characterized by the fact that the liquid phase additionally contains at least one cosmetic or dermatological active substance or care and maintenance substance, which is selected from among
I. natural, if desired, chemically modified, polymers, selected from among the following: cellulose ethers, quaternized cellulose derivatives, polyquaternium 24, guar gum, cationic guar derivatives, alginates, xanthan gum, gum arabic, karaya gum, carob bean flour, linseed gums, dextrans, shellac, amylose, amylopectin, dextrins, chemically and/or thermally modified starches, and chitosan and its derivatives,
II. synthetic polymers that do not act as superabsorbers but rather swell with water and are thereby converted to a gel-like true or colloidal solution,
III. o-hydroxycarboxylic acids and their derivatives.
IV. vitamins, provitamins, and vitamin intermediates of the groups B, C, and H and their derivatives,
V. plant extracts, selected from among plant formative tissues that are capable of dividing (meristem), green tea (Camellia sinensis), hamamelis, chamomile, marigold, pansy, peony, horse chestnut, sage, willow bark, cinnamon tree, chrysanthemums, oak bark, nettle, hops, lappa, horsetail, hawthorn, linden flowers, almonds, spruce needles, sandalwood, juniper, coconut, kiwi, guava, lime, mango, apricot, wheat, melon, orange, grapefruit, avocado, rosemary, birch, beech sprouts, mallow, cuckoo flower, yarrow, wild thyme, thyme, balm mint, resthallow, marsh mallow (Althaea), common mallow (Malva sylvestris), violet, leaves of the black currant, coltsfoot, cinquefoil, ginseng, ginger root, sweet potato, olives (Olea europaea), and citrus fruit seeds,
VI. extracts of algae and microorganisms,
VII. active substances with antiperspirant activity, selected from among astringent water-soluble inorganic and organic aluminum, zinc, and zirconium salts and their mixtures,
VIII. active substances with deodorant activity,
IX. silicic acids, natural and synthetic silicates, aluminosilicates, kaolin, talc, and apatites, which may be modified with aqueous carboxylic acids with 2-3 C atoms.
X. pigments selected from among the oxides of titanium, iron, zinc, zirconium, cerium, magnesium, and bismuth, which, if desired, may be surface-modified, boron nitride particles, water-insoluble nacreous pigments, and water-insoluble organic pigments,
XI. water-soluble and oil-soluble organic sunscreening agents.
XII. cosmetic abrasives selected from among polymer particles and vegetable abrasives, which, if desired, may be coated with fatty substances.
XIII. dyes and oxidation dye (intermediates) for dyeing keratinous fibers,
XIV. oxidizing and reducing agents, and
XV. active substances with sebum-regulating, skin-soothing, anti-inflammatory, astringent, or perfusion-promoting activity.

2. Use of a support material in accordance with claim 1 as a foam pad for face and body cleansing or for makeup removal.

3. Use of a support material in accordance with claim 1 as a 2-in-1 foam pad for cleansing and simultaneous care of the skin of the face and/or body.

4. Use of a support material in accordance with claim 1 as a skin-care foam pad.

5. Use of a support material water foam pad or tonic water foam the skin of the face and/or body.

6. Use of a support material in accordance application of a sunscreen on the skin.

7. Use of a support material in accordance foam pad.

8. Use of a support material in accordance with claim 1 as an anti-acne foam pad or as a disinfectant foam pad.

9. Use of a support material in accordance with claim 1 as a shampooing and hair-conditioning foam pad.

10. Use of a support material in accordance with claim 1 as an after-shave foam pad.

11. Use of a support material in accordance with claim 1 as a perfume foam pad.

12. Use of a support material in accordance with claim 1 as a foam pad for the application of rouge or makeup.

13. Use of a support material in accordance with claim 1 as a hair-dyeing foam pad and/or a hair strand foam pad for dyeing selected strands of hair.

14. Use of a support material in accordance with claim 1 as an antiperspirant foam pad.

15. Use of a support material in accordance with claim 1 as a deodorant foam pad.
